SEQUENCE LISTING (26 sequences)

SEQ ID NO: 1 - DNA, 54 bp, synthetic construct
  source 1..54: mol_type = other DNA
  misc_structure 27..28: note = Indocarbocyanine
cgccatcctagccgctatgtcattganncgagctgtcgagcatgtgagcattga

SEQ ID NO: 2 - DNA, 52 bp, synthetic construct
  source 1..52: mol_type = other DNA
ggcaatcagatcgacgtgacgctacagagcagtctatcaggcgagttggtcg

SEQ ID NO: 3 - DNA, 20 bp, synthetic construct
  source 1..20: mol_type = other DNA
tcaattgctctgtaggctcg

SEQ ID NO: 4 - DNA, 20 bp, synthetic construct
  source 1..20: mol_type = other DNA
cgaccaactctaggatgacg

SEQ ID NO: 5 - DNA, 22 bp, synthetic construct
  source 1..22: mol_type = other DNA
tagacgacatagcggcgcctga

SEQ ID NO: 6 - DNA, 22 bp, synthetic construct
  source 1..22: mol_type = other DNA
gtcgacatgctcgacacgtcac

SEQ ID NO: 7 - DNA, 20 bp, synthetic construct
  source 1..20: mol_type = other DNA
tcaatgctcatctgattgcc

SEQ ID NO: 8 - DNA, 53 bp, synthetic construct
  source 1..53: mol_type = other DNA
ggcaatcagatcgacgtgacgctacaggagcagtctatcaggcgagttggtcg

SEQ ID NO: 9 - DNA, 21 bp, synthetic construct
  source 1..21: mol_type = other DNA
tcaattgctcctgtaggctcg

SEQ ID NO: 10 - DNA, 54 bp, synthetic construct
  source 1..54: mol_type = other DNA
ggcaatcagatcgacgtgacgctacatggagcagtctatcaggcgagttggtcg

SEQ ID NO: 11 - DNA, 22 bp, synthetic construct
  source 1..22: mol_type = other DNA
tcaattgctccatgtaggctcg

SEQ ID NO: 12 - DNA, 55 bp, synthetic construct
  source 1..55: mol_type = other DNA
ggcaatcagatcgacgtgacgctacactggagcagtctatcaggcgagttggtcg

SEQ ID NO: 13 - DNA, 23 bp, synthetic construct
  source 1..23: mol_type = other DNA
tcaattgctccagtgtaggctcg

SEQ ID NO: 14 - DNA, 56 bp, synthetic construct
  source 1..56: mol_type = other DNA
ggcaatcagatcgacgtgacgctacacctggagcagtctatcaggcgagttggtcg

SEQ ID NO: 15 - DNA, 24 bp, synthetic construct
  source 1..24: mol_type = other DNA
tcaattgctccaggtgtaggctcg

SEQ ID NO: 16 - DNA, 57 bp, synthetic construct
  source 1..57: mol_type = other DNA
ggcaatcagatcgacgtgacgctacaacctggagcagtctatcaggcgagttggtcg

SEQ ID NO: 17 - DNA, 25 bp, synthetic construct
  source 1..25: mol_type = other DNA
tcaattgctccaggttgtaggctcg

SEQ ID NO: 18 - DNA, 50 bp, synthetic construct
  source 1..50: mol_type = other DNA
ggcaatcagatcgacgtgacgctacagcagtctatcaggcgagttggtcg

SEQ ID NO: 19 - DNA, 18 bp, synthetic construct
  source 1..18: mol_type = other DNA
tcaattgctgtaggctcg

SEQ ID NO: 20 - DNA, 49 bp, synthetic construct
  source 1..49: mol_type = other DNA
ggcaatcagatcgacgtgacgctaagcagtctatcaggcgagttggtcg

SEQ ID NO: 21 - DNA, 17 bp, synthetic construct
  source 1..17: mol_type = other DNA
tcaattgcttaggctcg

SEQ ID NO: 22 - DNA, 48 bp, synthetic construct
  source 1..48: mol_type = other DNA
ggcaatcagatcgacgtgacgctagcagtctatcaggcgagttggtcg

SEQ ID NO: 23 - DNA, 16 bp, synthetic construct
  source 1..16: mol_type = other DNA
tcaattgctaggctcg

SEQ ID NO: 24 - DNA, 47 bp, synthetic construct
  source 1..47: mol_type = other DNA
ggcaatcagatcgacgtgacgctgcagtctatcaggcgagttggtcg

SEQ ID NO: 25 - DNA, 15 bp, synthetic construct
  source 1..15: mol_type = other DNA
tcaattgcaggctcg

SEQ ID NO: 26 - DNA, 15 bp, synthetic construct
  source 1..15: mol_type = other DNA
ctacaacctggagca

(19) United States
(12) Patent Application Publication
(10) Pub. No.: US 2023/0295695 A1
(43) Pub. Date: Sep. 21, 2023

(54) DNA-DYE ASSEMBLY BASED SINGLE-MOLECULE FLUORESCENCE LIFETIME IMAGING PROBES

(71) Applicant: Massachusetts Institute of Technology, Cambridge, MA (US)

(72) Inventors: Stephanie Marie Hart, Cambridge, MA (US); Xiao Wang, Cambridge, MA (US); Mark Bathe, Cambridge, MA (US); Gabriela S. Schlau-Cohen, Cambridge, MA (US); Jiajia Guo, Changchun (CN)

(21) Appl. No.: 18/051,344

(22) Filed: Oct. 31, 2022

Related U.S. Application Data
(60) Provisional application No. 63/274,457, filed on Nov. 1, 2021.

(51) Int. Cl. C12Q 1/6832; C12Q 1/6876

(52) U.S. Cl. CPC C12Q 1/6832; C12Q 1/6876; C12Q 2600/16

(57) ABSTRACT

Disclosed are nucleic acid-chromophores and nucleic acid assembly containing the nucleic acid-chromophores. The nucleic acid-chromophore contains a nucleic acid strand and two or more adjacent chromophores. The two or more adjacent chromophores can be covalently incorporated in the backbone of the nucleic acid strand. One or more photophysical properties of the adjacent chromophores can be altered by a change in the nucleic acid assembly. In some forms, the nucleic acid assembly can contain a nucleic acid scaffold. In these forms, the change in the nucleic acid assembly can be a change in the length of a nucleic acid hybrid in the nucleic acid scaffold that is opposite the adjacent chromophores. Typically, the nucleic acid hybrid does not contain any chromophore. The nucleic acid-chromophores can serve as molecular fluorophores with emission properties that are highly sensitive to local geometry and the chemical environment.

### CROSS-REFERENCE TO RELATED APPLICATION

This application claims the benefit of and priority to U.S. Provisional Application No. 63/274,457 filed Nov. 1, 2021, which is hereby incorporated by reference in its entirety.

### STATEMENT REGARDING FEDERALLY SPONSORED RESEARCH

This invention was made with government support under Grant Numbers DE-SC0019998 awarded by the Department of Energy, 1839155 awarded by the National Science Foundation, GM128200 awarded by the National Institutes of Health and N00014-21-1-4013 awarded by the US Navy. The U.S. government has certain rights in this invention.

### REFERENCE TO SEQUENCE LISTING

The Sequence Listing submitted on Oct. 31, 2022, as a text file named “MIT_23780_ST26.xml,” created on Oct. 4, 2022, and having a size of 24,633 bytes is hereby incorporated by reference pursuant to 37 C.F.R § 1.52(e)(5).

### FIELD OF THE INVENTION

This invention is generally directed to nucleic acid assembly-based imaging probes.

### BACKGROUND OF THE INVENTION

The use of molecular systems to harness and direct energy from light underlies a wide range of phenomena including imaging, molecular sensing, computation, and photovoltaic conversion. However, a significant barrier for the use of optically addressable molecular systems, particularly for imaging, lies in the challenges associated with tuning their photophysical properties. Some level of tunability was reported through nuclear modification of monomeric chromophores. However, this process requires complex chemical synthesis routes, and the resulting changes in photophysical properties, if any, cannot be straightforwardly predicted. Alternatively, modulation of dimeric intermolecular interactions results in dramatic changes in photophysical properties owing to the steep distance and orientation dependence of dipole-dipole interactions (Scholes et al. Nature Materials 2006, 5, 683-696), but with this extensive tunability comes stringent structural requirements.

DNA origami can be used to generate arbitrary three-dimensional structures with nanoscale precision (Wamhoff et al. Annual Review of Biophysics 2019, 48, 395-419). A large toolkit of modified bases allows for site-specific conjugation of molecules within the DNA structure, as well as integration of these constructs into larger-scale devices (Markova et al. Chemical Communications 2013, 49, 5298-5300; Garo et al. Angewandte Chemie 2012, 51, 916-919). Through this capability, molecular chromophores can be scaffolded within DNA in precise and programmable spatial organizations (Boulais et al. Nature Materials 2018, 17, 159-166; Huff, et al., The Journal of Physical Chemistry B 2021, ASAP). The use of DNA-chromophore assembles is wide ranging and includes application in chemical sensory assays, and fluorescence imaging. Fluorescent nucleic acids have been previously used as optical sensors for metal ions, small molecules, and biomarkers (Yang, et al., ACS Sensors 2018, 3, 903-919; Tan et al. Journal of the American Chemical Society 2011, 133, 2664-2671). In addition, optical switches, imaging probes, and multiplexed detection assays have been generated through strand displacement reactions (Kellis, et al., New Journal of Physics 2015, 17, 115007; Tseng, et al., Journal of Biomedical Optics 2013, 18, 101309; Guo, et al., Nature Communications 2019, 10, 1-14).

Despite the potential of this approach, DNA-chromophore constructs can only be produced in a few geometries, limiting the utility of this synthetic platform. Covalent methods of attachment include nucleoside modification and direct incorporation into the DNA backbone, thus providing increased control over the nature of the excitonic (and thus delocalized) states formed (Knowlton, et al., U.S. patent application Ser. No. 16/739,963; Ensslen, et al., Accounts of Chemical Research 2015, 48, 2724-2733; Vybornyi, et al., Bioconjugate Chemistry, 2014, 25, 1785-1793). The later approach has been recently expanded upon toward the construction of molecular dimers and trimers with variable electronic coupling (Hart, et al., Chem 2021, 7, 752-772). However, these methods of controlling the resultant exciton properties typically rely on modifications to the chromophore containing DNA strands, which require complex syntheses and have limited tunability in their hybridized form.

There remains a need for nucleic acid-chromophores having a structure that allows for alteration of photophysical property(ies) by a simple change in the construct structure.

Therefore, it is the object of the present invention to provide nucleic acid-chromophores having photophysical properties that can be altered by a change in a nucleic acid assembly containing the nucleic acid-chromophores.

It is another object of the present invention to provide methods of making the nucleic acid-chromophores.

It is yet another object of the present invention to provide methods of using the nucleic acid-chromophores.

### SUMMARY OF THE INVENTION

Nucleic acid-chromophores and nucleic acid assembly containing the nucleic acid-chromophores have been developed. The nucleic acid-chromophores described herein can serve as molecular fluorophores with emission properties that are highly sensitive to local geometry and the chemical environment. For example, the nucleic acid-chromophores can be useful for generating DNA-based fluorescence lifetime imaging (FLIM) probes for applications such as imaging, multiplexed measurements, tracking strand displacement reactions, and monitoring DNA folding processes. They can also be useful nanoscale light harvesters and molecular electronics.

The nucleic acid-chromophore contains a nucleic acid strand and two or more adjacent chromophores. The two or more adjacent chromophores can be incorporated in the backbone of the nucleic acid strand, optionally covalently incorporated in the backbone of the nucleic acid strand. One or more photophysical properties of the adjacent chromophores can be altered by a change in the nucleic acid assembly.

In some forms, the nucleic acid assembly containing the nucleic acid-chromophore can contain a nucleic acid scaffold. In these forms, the change in the nucleic acid assembly can be a change in the length of a nucleic acid hybrid in the nucleic acid scaffold that is opposite the adjacent chromophores. In some forms, the nucleic acid assembly can contain a double crossover (“DX”) tile and the change in the nucleic acid assembly can be a change in the length of a nucleic acid hybrid in the DX tile opposite the adjacent chromophores. Typically, the nucleic acid hybrid does not contain any chromophore.

In some forms, one or more of the chromophores of the nucleic acid-chromophore can be a cyanine, a squaraine, a pentacene, or a perylene diimide. In some forms, one or more of the chromophores of the nucleic acid-chromophore can be a cyanine. In some forms, all of the adjacent chromophores of the nucleic acid-chromophore can be cyanines.

In some forms, the nucleic acid strand of the nucleic acid-chromophore can be composed of deoxyribonucleotides (DNA), ribonucleotides (RNA), or analogs or modified nucleotides thereof. In some forms, the nucleic acid strand of the nucleic acid-chromophore can be composed of deoxyribonucleotides (DNA). In some forms, the nucleic acid strand of the nucleic acid-chromophore can be composed of locked nucleic acids (LNA) or peptide nucleic acids (PNA).

In some forms, the nucleic acid-chromophore can be in a nucleic acid assembly, wherein the nucleic acid assembly can be coupled to a biological or nonbiological material. In some forms, the nucleic acid assembly can be operably coupled to the biological or nonbiological material, wherein interaction of a molecule of interest with the biological or nonbiological material produces the change in the nucleic acid assembly.

In some forms, the one or more photophysical properties of the adjacent chromophores of the nucleic acid-chromophore includes the quantum yield of the adjacent chromophores. In some forms, the one or more photophysical properties of the adjacent chromophores of the nucleic acid-chromophore includes the energy dependent optical density of the adjacent chromophores. In some forms, the one or more photophysical properties of the adjacent chromophores of the nucleic acid-chromophore includes the emission energetic profile of the adjacent chromophores. In some forms, the one or more photophysical properties of the adjacent chromophores of the nucleic acid-chromophore includes the excited state lifetime of the adjacent chromophores. In some forms, the one or more photophysical properties of the adjacent chromophores of the nucleic acid-chromophore includes the excited state lifetime of the adjacent chromophores, wherein the excited state lifetime of the adjacent chromophores can be altered by a change in solvent polarity. In some forms, the alteration in the one or more photophysical properties of the adjacent chromophores of the nucleic acid-chromophore can be sufficient to distinguish a single altered nucleic acid assembly from a single unaltered nucleic acid assembly.

In some forms, the nucleic acid assembly containing the nucleic acid-chromophore can be encapsulated. In some forms, the nucleic acid assembly containing the nucleic acid-chromophore can be encapsulated in an organic or inorganic material. In some forms, the nucleic acid assembly containing the nucleic acid-chromophore can be encapsulated in an organic material. In some forms, the nucleic acid assembly containing the nucleic acid-chromophore can be encapsulated in an inorganic material. When the nucleic acid assembly containing the nucleic acid-chromophore is encapsulated in an inorganic material, the inorganic material can be silica.

Methods of detecting a change in a nucleic acid assembly, wherein the nucleic acid assembly contains the nucleic acid-chromophore described herein, are disclosed. The method can include measuring one or more of the photophysical properties of the adjacent chromophores, wherein the change can be detected if one or more of the measured photophysical properties of the adjacent chromophores is altered compared to a reference photophysical property. In some forms, the reference photophysical property can be the photophysical property of the nucleic acid assembly in the absence of a change in the nucleic acid assembly. In some forms, the change in the nucleic acid assembly can be produced by interaction of a molecule of interest with a biological or nonbiological material to which the nucleic acid assembly is operably coupled. In some forms, the change in the nucleic acid assembly can be produced by correct assembly of a scaffold origami of which the nucleic acid assembly becomes a part. In some forms, the change in the nucleic acid assembly can be produced by a change in the milieu of the nucleic acid assembly. In some forms, the change in milieu of the nucleic acid assembly can be a change in the polarity of solvent in which the nucleic acid assembly is dissolved or suspended.

Methods of multiplex detection are disclosed. The method can include (i) labeling each of a first plurality of different targets of interest with the same first nucleic acid-chromophore described herein, wherein each of the first nucleic acid-chromophores labeling each of the plurality of different targets of interest can be in a different nucleic acid assembly, wherein each of the different nucleic acid assemblies can have a change relative to the other nucleic acid assemblies such that one or more of the photophysical properties of the adjacent chromophores can be altered relative to those photophysical properties of the adjacent chromophores in the other nucleic acid assemblies, and (ii) detecting the photophysical properties of the adjacent chromophores in the different nucleic acid assemblies, thereby detecting the different targets of interest of the first plurality of different targets of interest. In some forms, the method also includes a step of labeling each of a second plurality of different targets of interest with the same second nucleic acid-chromophore described herein, wherein each of the second nucleic acid-chromophores labeling each of the second plurality of different targets of interest can be in a different nucleic acid assembly, wherein each of the different nucleic acid assemblies can have a change relative to the other nucleic acid assemblies such that one or more of the photophysical properties of the adjacent chromophores can be altered relative to those photophysical properties of the adjacent chromophores in the other nucleic acid assemblies, and detecting the photophysical properties of the adjacent chromophores in the different nucleic acid assemblies, thereby detecting the different targets of interest of the second plurality of different targets of interest.

Methods of altering one or more photophysical properties of the nucleic acid-chromophore described herein are also disclosed. The method can include making a change in a nucleic acid assembly, wherein the nucleic acid assembly can contain any nucleic acid chromophore described herein. The one or more photophysical properties of the nucleic acid-chromophore can be altered by the change in the nucleic acid assembly.

### BRIEF DESCRIPTION OF THE DRAWINGS

FIGS. 1A-1K are graphs and schematics showing the electronic transitions of Cy3 dimers upon the addition or removal of central crossover strand base. FIG. 1A shows the chemical structure of the Cy3 dimer. FIG. 1B shows a schematic of Cy3 dimer that is covalently bridged within a DX tile. FIGS. 1C-1D show a schematic and sequences for Cy3-DX tiles upon the addition (FIG. 1C, “pull”-type, with the nucleic acid sequence CTACAACCTGGAGCA (SEQ ID NO:26) shown spanning the region from +1 to =5 bp) and removal (FIG. 1D, “push”-type, with the nucleic acid sequence CTGCA shown spanning the region from −2 to −5 bp) of staple strand bases. FIGS. 1E-1F show the absorption (solid) and emission (dashed) spectra of “pull”-type (FIG. 1E) and “push”-type (FIG. 1F) dimers. Emission spectra are normalized by relative quantum yield. FIGS. 1G-1H show the circular dichroism spectra of “pull”-type (FIG. 1G) and “push”-type (FIG. 1H) dimers. FIGS. 1I-1K show the steady state absorption and emission properties: peak molar absorptivity (FIG. 1I), ratio between v00 and v01 absorption bands (FIG. 1J), and quantum yield (FIG. 1K) as a function of nucleotide spacing (monomer also included). The values are summarized in Tables 3 and 4.

FIGS. 2A-2D are schematics showing the construction scheme for the DX tile dimer based fluorophore construct. FIG. 2A shows the work flow for strand synthesis and construct generation. FIG. 2B shows the strand structure of the dimer before changes. FIG. 2C shows the changes in strand structure of the “pull type” dimer upon the addition of bases to strands B* and C*. FIG. 2D shows the changes in strand structure of the “push type” dimer upon the removal of bases from strands B* and C*.

FIGS. 3A-3C are graphs showing the fluorescence lifetime upon the addition or removal of central crossover strand bases. Fluorescence decay traces with 520 nm excitation for dimers formed with the addition or “push”-type (FIG. 3A) and removal or “pull”-type (FIG. 3B), of central crossover strand bases. FIG. 3C shows the average emission lifetimes extracted from the decay curves in FIGS. 3A-3B.

FIG. 4 is a graph showing the monomer TCSPC lifetime, upon 520 nm excitation and 580 nm emission. The values are summarized in Table 5.

FIGS. 5A-5D are graphs showing the ultrafast transient absorption spectra identify two subpopulations. FIGS. 5A-5B shows the broadband transient absorption spectra of 0 bp dimer (FIG. 5A) and +4 bp dimer (FIG. 5B). FIG. 5C shows the transient absorption kinetic traces from FIGS. 5A and 5B at 19,800 cm-1 and 17,600 cm-1 for 0 bp (purple) and +4 bp dimer (red). FIG. 5D shows the globally fit decay associated spectra (DAS) of FIGS. 5A and 5B for 0 bp and +4 bp. The long timescale is shown in solid lines (τ=4 ns and τ=1.7 ns for 0 and +4 bp, respectively) and the short timescale is shown in dashed lines (τ=145 ps and τ=330 ps for 0 and +4 bp, respectively). The +4 bp DAS is scaled by a factor of 1.7 for ease of visualization.

FIG. 6A-6D are graphs showing the solvent dependent emission spectra: the excitation and emission spectra re-covered for 624 nm emission and 520 nm excitation in buffered solvent of water and 30% DMF/70% water mixture for 0 bp dimer (FIG. 6A), +4 bp dimer (FIG. 6B), dimer containing single-stranded DNA (FIG. 6C), and Cy3 monomer containing DX tile (FIG. 6D). All spectra are normalized to their maximum value.

FIGS. 7A-7C are graphs showing the emission changes with environment polarity. FIG. 7A shows the change in excitation spectrum under addition of 30% DMF (relative to area normalized spectrum in 0% DMF). Dashed lines indicates the 0-0 and 0-1 transitions for the 0 bp dimer. FIG. 7B shows the average emission lifetimes upon 545 nm excitation for Cy3 dimers within 0 bp and +4 bp DX tiles and single-stranded DNA, and Cy3 monomers within 0 bp DX tiles in 0% DMF and 30% DMF. FIG. 7C shows the difference in average lifetime (30% DMF-0% DMF) for DNA constructs.

FIGS. 8A-8F are graphs showing the single-molecule imaging of Cy3-DNA constructs. FIGS. 8A, 8C, and 8E show the distribution of single-molecule emission lifetimes for immobilized samples of 0 bp dimers (FIG. 8A), +4 bp dimers (FIG. 8C), and a 50% mixture of 0 bp and +4 bp dimers (FIG. 8E). Dashed lines indicate the mean of the 0 bp and +4 bp distributions. FIGS. 8B, 8D, and 8F show the phasor plots calculated according to ŠStefl, et al., Analytical Biochemistry, 410:62-69 (2011) for 0 bp dimers (FIG. 8B), +4 bp dimers (FIG. 8D), and a 50% mixture of 0 bp and +4 bp dimers (FIG. 8F).

FIGS. 9A-9B are graphs showing the spectral and temporal characterization for transient absorption spectroscopy. FIG. 9A shows the SHG derived temporal correlation between pump and probe pulses. Gaussian fit with 190 fs FWHM show in gray. FIG. 9B shows the spectral profile of compressed pump pulse derived from a transient grating frequency resolved optical gating experiment.

FIG. 10 is a graph showing the monomer CD optical response in comparison to 0 bp dimer.

FIGS. 11A-11B are graphs showing the TCSPC time domain traces: time correlated single photon counting trace for 520 nm excitation for DX tiles upon the addition (FIG. 11A) and removal (FIG. 11B) of base pairs. Fits shown in black.

FIGS. 12A-12D are bar graphs showing the trends in globally fit TCSPC lifetimes upon fitting the short timescale globally to 0.25 ns and 0.2 ns for the addition and removal of base pairs, respectively: fractional short lifetime component for dimers formed upon the addition (FIG. 12A) and removal (FIG. 12C) of complementary base pairs, and fractional short lifetime component for dimers formed upon the addition (FIG. 12B) and removal (FIG. 12D) of complementary base pairs.

FIGS. 13A-13B are graphs showing the excitation spectra for 630 nm emission: excitation spectrum variation under the addition (FIG. 13A) and subtraction (FIG. 13B) of complementary base pairs.

FIGS. 14A-14D are graphs showing the solvent dependent emission timescales: TCSPC recovered emissive lifetime for 545 nm excitation in buffered solvent of water and 30% DMF/70% water mixture of 0 bp dimer (FIG. 14A), +4 bp dimer (FIG. 14B), dimer containing single-stranded DNA (FIG. 14C), and Cy3 monomer containing DX tile (FIG. 14D).

FIGS. 15A-15D are graphs showing the solvent dependent emission spectra: excitation and emission spectra re-covered for 624 nm emission and 520 nm excitation in buffered solvent of water and 30% DMF/70% water mixture for 0 bp dimer (FIG. 15A), +4 bp dimer (FIG. 15B), dimer containing single-stranded DNA (FIG. 15C), and Cy3 monomer containing DX tile (FIG. 15D). All spectra are normalized to their maximum value.

FIGS. 16A-16D are graphs showing the solvent dependent absorption: absorption spectra for buffered solvent of water and 30% DMF/70% water mixture corresponding to 0 bp dimer (FIG. 16A), +4 bp dimer (FIG. 16B), dimer containing single-stranded DNA (FIG. 16C), and Cy3 monomer containing DX tile (FIG. 16D).

FIGS. 17A-17D are graphs showing the time and frequency traces recovered from global analysis. FIGS. 17A and 17C show the spectra recovered from 0 bp (FIG. 17A) and +4 bp (FIG. 17C) transient absorption data (purple and orange, respectively) and corresponding fits from global analysis of the independent decays (gray). Solid and dashed lines corresponding to population times of 30 ps and 400 ps, respectively, after photoexcitation. FIGS. 17B and 17D show the temporal traces recovered from 0 bp (FIG. 17B) and +4 bp (FIG. 17D) transient absorption data (purple and orange, respectively) and corresponding traces from global analysis of the independent decays (gray). Solid and dashed lines correspond to probe frequencies of 20,000 cm“1 and 18,300 cm”1, respectively.

FIGS. 18A-18B are graphs showing the representative fluorescence traces of single molecules: single-molecule traces for 0 bp (FIG. 18A) and +4 bp DX tile dimers (FIG. 18B).

FIGS. 19A-19C are graphs showing the silicification-induced lifetime differences in six helix bundles. FIG. 19A shows a schematic of a 6-helix bundle-based triangle DNA origami scaffolding a dimeric cyanine. FIG. 19B shows an AFM image of the construct in FIG. 19A. FIG. 19C shows a comparison of fluorescence lifetimes of the construct in FIG. 19A in silica and buffer.

### DETAILED DESCRIPTION OF THE INVENTION

### I. Definitions

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

“Alkyl,” as used herein, refers to the radical of saturated aliphatic groups, including straight-chain alkyl groups, branched-chain alkyl, and cycloalkyl (alicyclic). In some forms, a straight chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non-aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

The term “alkyl” as used throughout the specification, examples, and claims is intended to include both “unsubstituted alkyls” and “substituted alkyls,” the latter of which refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety. —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF₃, —CH₂—CF₃, —CCl₃); —CN; —NCOCOCH₂CH₂; —NCOCOCHCH; and —NCS; and combinations thereof. The term “alkyl” also includes “heteroalkyl.”

It will be understood by those skilled in the art that the moieties substituted on the hydrocarbon chain can themselves be substituted, if appropriate. For instance, the substituents of a substituted alkyl may include halogen, hydroxy, nitro, thiols, amino, aralkyl, azido, imino, amido, phosphonium, phosphanyl, phosphoryl (including phosphonate and phosphinate), oxo, sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

“Heteroalkyl,” as used herein, refers to straight or branched chain, or cyclic carbon-containing alkyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure. Cycloalkenyls containing a polycyclic ring system can have two or more non-aromatic rings in which two or more carbons are common to two adjoining rings (i.e., “fused cycloalkenyl rings”) and contain at least one carbon-carbon double bond. Asymmetric structures such as (AB)C=C(C′D) are intended to include both the E and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl.”

The term “substituted alkenyl” refers to alkenyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g., quarternized amino), amido, amidine, imine, cyano, nitro, azido, oxo, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond. Asymmetric structures such as (AB)C≡C(C″D) are intended to include both the E and Z isomers. This may be presumed in structural formulae herein wherein an asymmetric alkyne is present, or it may be explicitly indicated by the bond symbol C. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl.”

The term “substituted alkynyl” refers to alkynyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkynyl,” as used herein, refers to straight or branched chain, or cyclic carbon-containing alkynyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkynyl group” is a cycloalkynyl group where at least one of the carbon atoms of the ring is substituted with a heteroatom such as, but not limited to, nitrogen, oxygen, sulphur, or phosphorus.

The term “aryl” as used herein is any C₅-C₂₆ carbon-based aromatic group, heteroaromatic, fused aromatic, or fused heteroaromatic. For example, “aryl,” as used herein can include 5-, 6-, 7-, 8-, 9-, 10-, 14-, 18-, and 24-membered single-ring aromatic groups, including, but not limited to, benzene, naphthalene, anthracene, phenanthrene, chrysene, pyrene, corannulene, coronene, etc. “Aryl” further encompasses polycyclic ring systems having two or more cyclic rings in which two or more carbons are common to two adjoining rings (i.e., “fused aromatic rings”), wherein at least one of the rings is aromatic, e.g., the other cyclic ring or rings can be cycloalkyls, cycloalkenyls, cycloalkynyls, aryls and/or heterocycles. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “substituted aryl” refers to an aryl group, wherein one or more hydrogen atoms on one or more aromatic rings are substituted with one or more substituents. Such substituents can be any substituents described above, e.g., halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, imino, alkylthio, sulfate, sulfonate, sulfamoyl, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocycle” and “heterocyclyl” are used interchangeably, and refer to a cyclic radical attached via a ring carbon or nitrogen atom of a non-aromatic monocyclic or polycyclic ring containing 3-30 ring atoms, 3-20 ring atoms, 3-10 ring atoms, or 5-6 ring atoms, where each ring contains carbon and one to four heteroatoms each selected from the group including non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc, such as piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₃₀-membered aromatic, fused aromatic, biaromatic ring systems, or combinations thereof, in which one or more carbon atoms on one or more aromatic ring structures have been substituted with a heteroatom. Suitable heteroatoms include, but are not limited to, oxygen, sulfur, and nitrogen. Broadly defined, “heteroaryl,” as used herein, includes 5-, 6-, 7-, 8-, 9-, 10-, 14-, 18-, and 24-membered single-ring aromatic groups that may include from one to four heteroatoms, for example, pyrrole, furan, thiophene, imidazole, oxazole, thiazole, triazole, tetrazole, pyrazole, pyridine, pyrazine, pyridazine and pyrimidine, and the like. The heteroaryl group may also be referred to as “aryl heterocycles” or “heteroaromatics.” “Heteroaryl” further encompasses polycyclic ring systems having two or more rings in which two or more carbons are common to two adjoining rings (i.e., “fused rings”) wherein at least one of the rings is heteroaromatic, e.g., the other cyclic ring or rings can be cycloalkyls, cycloalkenyls, cycloalkynyls, aryls, heterocycles, or combinations thereof. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The term “substituted heteroaryl” refers to a heteroaryl group in which one or more hydrogen atoms on one or more heteroaromatic rings are substituted with one or more substituents. Such substituents can be any substituents described above, e.g., halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, imino, alkylthio, sulfate, sulfonate, sulfamoyl, sulfonamido, sulfonyl, heterocyclyl, alkylaryl, haloalkyl (such as CF₃, —CH₂—CF₃, —CCl₃), —CN, aryl, heteroaryl, and combinations thereof.

The term “polyaryl” refers to a chemical moiety that includes two or more aryls, heteroaryls, and combinations thereof. The aryls, heteroaryls, and combinations thereof, are fused, or linked via a single bond, ether, ester, carbonyl, amide, sulfonyl, sulfonamide, alkyl, azo, and combinations thereof. For example, a “polyaryl” can be polycyclic ring systems having two or more cyclic rings in which two or more carbons are common to two adjoining rings (i.e., “fused aromatic rings”), wherein two or more of the rings are aromatic.

When two or more heteroaryls are involved, the chemical moiety can be referred to as a “polyheteroaryl.”

The term “substituted polyaryl” refers to a polyaryl in which one or more of the aryls, heteroaryls are substituted, with one or more substituents. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g., quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. When two or more heteroaryls are involved, the chemical moiety can be referred to as a “substituted polyheteroaryl.”

The term “cyclo-” refers to a substituted or unsubstituted monocyclic ring or a substituted or unsubstituted polycyclic ring (such as those formed from single or fused ring systems), such as a substituted or unsubstituted cycloalkyl, a substituted or unsubstituted cycloalkenyl, a substituted or unsubstituted cycloalkynyl, a substituted or unsubstituted heterocyclyl, a substituted or unsubstituted aryl, a substituted or unsubstituted polyaryl, a substituted or unsubstituted heteroaryl, and a substituted or unsubstituted polyheteroaryl, that have from three to 30 carbon atoms, as geometric constraints permit. The substituted cycloalkyls, cycloalkenyls, cycloalkynyls, and heterocyclyls are substituted as defined above for the alkyls, alkenyls, alkynyls, heterocyclyls, aryls, heteroaryl, polyaryls, and polyheteroaryls, respectively.

The term “aralkyl” as used herein is an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The terms “alkoxyl” or “alkoxy,” “aroxy” or “aryloxy,” generally describe compounds represented by the formula —OR^{v}, wherein R^{v} includes, but is not limited to, a substituted or unsubstituted alkyl, a substituted or unsubstituted alkenyl, a substituted or unsubstituted alkynyl, a substituted or unsubstituted cycloalkyl, a substituted or unsubstituted heterocyclyl, a substituted or unsubstituted cycloalkenyl, a substituted or unsubstituted heterocycloalkenyl, a substituted or unsubstituted aryl, a substituted or unsubstituted heteroaryl, a substituted or unsubstituted polyaryl, a substituted or unsubstituted polyheteroaryl, a substituted or unsubstituted arylalkyl, a substituted or unsubstituted heteroalkyl, a substituted or unsubstituted alkylaryl, a substituted or unsubstituted alkylheteroaryl, a substituted or unsubstituted aralkyl, a substituted or unsubstituted carbonyl, a phosphonium, a phosphanyl, a phosphonyl, a sulfinyl, a silyl, a thiol, an amido, and an amino Exemplary alkoxyl groups include methoxy, ethoxy, propyloxy, tert-butoxy and the like. A “lower alkoxy” group is an alkoxy group containing from one to six carbon atoms. An “ether” is two functional groups covalently linked by an oxygen as defined below. Accordingly, the substituent of an alkyl that renders that alkyl an ether is or resembles an alkoxyl, such as can be represented by one of O alkyl, O alkenyl, O alkynyl, O aryl, O heteroaryl, O polyaryl, O polyheteroaryl, O heterocyclyl, etc.

The term “substituted alkoxy” refers to an alkoxy group having one or more substituents replacing one or more hydrogen atoms on one or more carbons of the alkoxy backbone. Such substituents can be any substituents described above, ., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, oxo, sulfate, sulfonate, sulfamoyl, sulfonamido, sulfonyl, heterocyclyl, alkylaryl, haloalkyl, —CN, aryl, heteroaryl, and combinations thereof.

The term “ether” as used herein is represented by the formula A²OA1, where A² and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a phosphonyl, a sulfinyl, a silyl, a thiol, a substituted or unsubstituted carbonyl, an alkoxy, an amido, or an amino, described above.

The term “polyether” as used herein is represented by the formula:

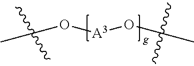

where A³, A², and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a substituted or unsubstituted carbonyl, an alkoxy, an amido, or an amino, described above; g can be a positive integer from 1 to 30.

The term “phenoxy” is art recognized and refers to a compound of the formula —OR″ wherein R^{v} is (i.e., —O—C₆H₅). One of skill in the art recognizes that a phenoxy is a species of the aroxy genus. A substituted phenoxy refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein. A “substituted aroxy” or “substituted aryloxy,” refers to —O-aryl or —O-heteroaryl, having one or more substituents replacing one or more hydrogen atoms on one or more ring atoms of the aryl and heteroaryl, as defined herein. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “amino” as used herein includes the group

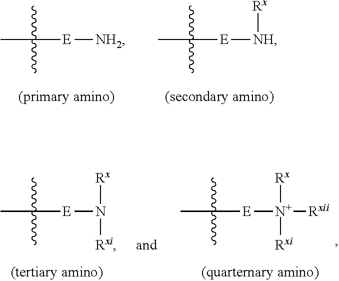

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl, wherein independently of E, R^{x}, R^{xi}, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. The term “quaternary amino” also includes the groups where the nitrogen, R^{x}, R^{xi}, and R^{xii} with the N⁺ to which they are attached complete a heterocyclyl or heteroaryl having from 3 to 14 atoms in the ring structure.

The terms “amide” or “amido” are used interchangeably, refer to both “unsubstituted amido” and “substituted amido” and are represented by the general formula:

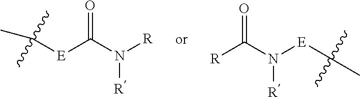

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted aryl, substituted or unsubstituted heteroaryl, a substituted or unsubstituted polyaryl, a substituted or unsubstituted polyheteroaryl, or a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. In some forms, when E is oxygen, a carbamate is formed.

“Carbonyl,” as used herein, is art-recognized and includes such moieties as can be represented by the general formula:

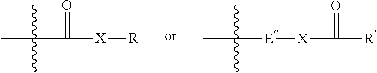

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″, or a pharmaceutical acceptable salt; E″ is absent, or E″ is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″; R″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Where X is oxygen and R is defined as above, the moiety is also referred to as a carboxyl group. When X is oxygen and R is hydrogen, the formula represents a “carboxylic acid.” Where X is oxygen and R′ is hydrogen, the formula represents a “formate.” Where X is oxygen and R or R′ is not hydrogen, the formula represents an “ester.” In general, where the oxygen atom of the above formula is replaced by a sulfur atom, the formula represents a “thiocarbonyl” group. Where X is sulfur and R or R′ is not hydrogen, the formula represents a “thioester.” Where X is sulfur and R is hydrogen, the formula represents a “thiocarboxylic acid.” Where X is sulfur and R′ is hydrogen, the formula represents a “thioformate.” Where X is a bond and R is not hydrogen, the above formula represents a “ketone.” Where X is a bond and R is hydrogen, the above formula represents an “aldehyde.”

A “substituted carbonyl” refers to a carbonyl, as defined above, wherein one or more hydrogen atoms in R, R′ or a group to which the moiety

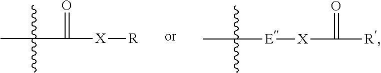

is attached, are independently substituted. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “carboxyl” is as defined above for carbonyl and is defined more specifically by the formula —R^{iv}COOH, wherein R^{iv} is a substituted or unsubstituted alkyl, a substituted or unsubstituted alkenyl, a substituted or unsubstituted alkynyl, a substituted or unsubstituted heterocyclyl, a substituted or unsubstituted alkylaryl, a substituted or unsubstituted arylalkyl, a substituted or unsubstituted aryl, a substituted or unsubstituted polyaryl, a substituted or unsubstituted polyheteroaryl, or a substituted or unsubstituted heteroaryl.

A “substituted carboxyl” refers to a carboxyl, as defined above, wherein one or more hydrogen atoms in R^{iv} are substituted. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “phosphanyl” is represented by the formula

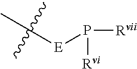

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi} and R^{vii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “phosphonium” is represented by the formula

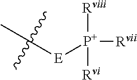

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi}, R^{vii}, and R^{viii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi}, R^{vii}, and R^{viii} taken together with the P⁺ atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “phosphonyl” is represented by the formula

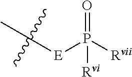

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, oxygen, alkoxy, aroxy, or substituted alkoxy or substituted aroxy, wherein, independently of E, R^{vi} and R^{vii} are independently a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

A “substituted phosphonyl” represents a phosphonyl in which E, R^{vi} and R^{vii} are independently substituted. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “phosphoryl” defines a phosphonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “sulfinyl” is represented by the formula

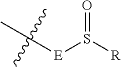

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a silyl, a thiol, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “sulfonyl” is represented by the formula

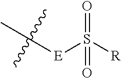

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

A “substituted sulfonyl” represents a sulfonyl in which E, R, or both, are independently substituted. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, a substituted or unsubstituted polyaryl, a substituted or unsubstituted polyheteroaryl, or substituted or unsubstituted heteroaryl.

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art.

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amino, substituted or unsubstituted cycloalkyl, substituted or unsubstituted heterocyclyl, substituted or unsubstituted aralkyl, substituted or unsubstituted alkylaryl, substituted or unsubstituted arylalkyl, substituted or unsubstituted aryl, substituted or unsubstituted heteroaryl, a substituted or unsubstituted polyaryl, a substituted or unsubstituted polyheteroaryl, —(CH₂)ₘ—R″, R′″ represents a hydroxy group, substituted or unsubstituted carbonyl group, an aryl, a cycloalkyl ring, a cycloalkenyl ring, a heterocycle, an amido, an amino, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art.

The term “sulfamoyl” refers to a sulfonamide or sulfonamide represented by the formula

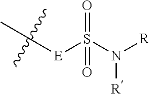

wherein E is absent, or E is substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted cycloalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The terms “thiol” are used interchangeably and are represented by —SR, where R can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above.

The term “alkylthio” refers to an alkyl group, as defined above, having a sulfur radical attached thereto. The “alkylthio” moiety is represented by —S-alkyl. Representative alkylthio groups include methylthio, ethylthio, and the like. The term “alkylthio” also encompasses cycloalkyl groups having a sulfur radical attached thereto. A “substituted alkylthio” refers to an alkylthio group having one or more substituents replacing one or more hydrogen atoms on one or more carbon atoms of the alkylthio backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “phenylthio” is art recognized, and refers to —S—C₆H₅, i.e., a phenyl group attached to a sulfur atom. A “substituted phenylthio” refers to a phenylthio group, as defined above, having one or more substituents replacing a hydrogen on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

“Arylthio” refers to —S-aryl or —S-heteroaryl groups, wherein aryl and heteroaryl as defined herein. A “substituted arylthio” represents —S-aryl or —S-heteroaryl, having one or more substituents replacing a hydrogen atom on one or more ring atoms of the aryl and heteroaryl rings as defined herein. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group including an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted with, independently, with the substituents described above in the definition of “substituted.”

The term “nucleotide” refers to a molecule that contains a base moiety, a sugar moiety and a phosphate moiety. Nucleotides can be linked together through their phosphate moieties and sugar moieties creating an inter-nucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The terms “nucleic acid,” “polynucleotide,” and “oligonucleotide” are interchangeable and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones, locked nucleic acid). In general and unless otherwise specified, an analogue of a particular nucleotide has the same base-pairing specificity; i.e., an analogue of A will base-pair with T. When double-stranded DNA is described, the DNA can be described according to the conformation adopted by the helical DNA, as either A-DNA, B-DNA, or Z-DNA. The B-DNA described by James Watson and Francis Crick is believed to predominate in cells, and extends about 34 Å per 10 bp of sequence; A-DNA extends about 23 Å per 10 bp of sequence, and Z-DNA extends about 38 Å per 10 bp of sequence.

In some cases nucleotide sequences are provided using character representations recommended by the International Union of Pure and Applied Chemistry (IUPAC) or a subset thereof. IUPAC nucleotide codes used herein include, A=Adenine, C=Cytosine, G=Guanine, τ=Thymine, U=Uracil, R=A or G, Y═C or T, S=G or C, W=A or T, K=G or T, M=A or C, B═C or G or T, D=A or G or T, H=A or C or T, V =A or C or G, N=any base, “.” or “-”=gap. In some forms the set of characters is (A, C, G, T, U) for adenosine, cytidine, guanosine, thymidine, and uridine respectively. In some forms the set of characters is (A, C, G, T, U, I, X, Ψ) for adenosine, cytidine, guanosine, thymidine, uridine, inosine, uridine, xanthosine, pseudouridine respectively. In some forms the set of characters is (A, C, G, T, U, I, X, Ψ, R, Y, N) for adenosine, cytidine, guanosine, thymidine, uridine, inosine, uridine, xanthosine, pseudouridine, unspecified purine, unspecified pyrimidine, and unspecified nucleotide respectively. The modified sequences, non-natural sequences, or sequences with modified binding, may be in the genomic, the guide or the tracr sequences.

The terms “scaffolded DNA origami,” “DNA origami” or “DNA nanostructure” are used interchangeably. They can refer to compositions and methods of using numerous short single strands of nucleic acids (staple strands) (e.g., DNA) to direct the folding of a long, single strand of polynucleotide (scaffold strand) into desired shapes on the order of about 10-nm to a micron or more, and the structures form therefrom.

The term “polyhedron” refers to a three-dimensional solid figure in which each side is a flat surface. These flat surfaces are polygons and are joined at their edges.

The terms “staple strands” or “helper strands” are used interchangeably. “Staple strands” or “helper strands” refer to oligonucleotides that hold the scaffold DNA in its three-dimensional wireframe geometry. Additional nucleotides can be added to the staple strand at either 5′ end or 3′ end, and those are referred to as “staple overhangs.” Staple overhangs can be functionalized to have desired properties such as a specific sequence to hybridize to a target nucleic acid sequence, or a targeting element. In some instances, the staple overhang is biotinylated for capturing the DNA nanostructure on a streptavidin-coated bead. In some instances, the staple overhang can be also modified with chemical moieties. Non-limiting examples include Click-chemistry groups (e.g., azide group, alkyne group, DIBO/DBCO), amine groups, and Thiol groups. In some instances some bases located inside the oligonucleotide can be modified using base analogs (e.g., 2-Aminopurine, Locked nucleic acids, such as those modified with an extra bridge connecting the 2′ oxygen and 4′ carbon) to serve as linker to attach functional moieties (e.g., lipids, proteins). Alternatively DNA-binding proteins or guide RNAs can be used to attach secondary molecules to the DNA scaffold.

The term “loop-crossover structure” refers to 3D structure in which endpoints are joined such that every duplex becomes part of a loop, and positions of possible scaffold double crossovers are found between two loops.

### II. Compositions

Nucleic acid-chromophores and nucleic acid assembly containing the nucleic acid-chromophores have been developed. The nucleic acid-chromophore contains a nucleic acid strand and two or more adjacent chromophores. The two or more adjacent chromophores can be covalently incorporated in the backbone of the nucleic acid strand. One or more photophysical properties of the adjacent chromophores can be altered by a change in the nucleic acid assembly.

In some forms, the nucleic acid assembly contains a nucleic acid scaffold. In these forms, the change in the nucleic acid assembly is a change in the length of a nucleic acid hybrid in the nucleic acid scaffold that is opposite the adjacent chromophores. In some forms, the nucleic acid assembly contains a double crossover (“DX”) tile and the change in the nucleic acid assembly is a change in the length of a nucleic acid hybrid in the DX tile opposite the adjacent chromophores. Typically, the nucleic acid hybrid does not contain any chromophore.

The nucleic acid-chromophores described herein can serve as molecular fluorophores with emission properties that are highly sensitive to local geometry and the chemical environment. For example, a DNA-cyanine dimer is incorporated in a DX tile, the building block of DNA origami assemblies. By adding or removing complementary bases in the nucleic acid hybrid, the DX tile “pushes” or “pulls” the cyanine dimer that is covalently bridged within the DX tile. This allows tuning of the electronic coupling, and thus the optical spectrum. The changes in the intradimer coupling lead to changes in one or more photophysical properties of the adjacent chromophores, such as the excited state lifetime. These mechanical changes allow the DNA-cyanine dimer incorporated DX tile to serve as a probe with a fluorescent readout to (1) structural changes of strands used to construct the DX tile, and (2) local environment changes, such as solvent polarity and encapsulation in silica. The data in the Examples also show that structural changes in these probes can be distinguishable on the single molecule level using confocal microscopy, which is a requirement for multiplexed imaging probes in techniques such as fluorescence lifetime imaging (FLIM). The tuning of inter-DX configuration is achieved by replacing unmodified DNA strands in the DNA assembly, which does not require the multi-step synthesis of the chromophore-containing strand.

These results demonstrate that the nucleic acid-chromophore described herein can be useful for generating DNA-based fluorescence lifetime imaging (FLIM) probes for applications such as imaging, multiplexed measurements, tracking strand displacement reactions, and monitoring DNA folding processes. They can also be useful nanoscale light harvesters and molecular electronics.

A. Nucleic Acid Assembly

The nucleic acid assembly typically includes at least a nucleic acid scaffold including a nucleic acid-chromophore formed of a nucleic acid scaffold strand including at least two chromophores hybridized to a nucleic acid hybrid, that depending on its length and sequence, can control the distance between the two chromophores. In some forms, nucleic acid assembly forms a higher order structure such as a polyhedral shape. Compositions and methods of making nucleic acid structures have been described in, for example, U.S. Published Application Nos. 2020/0327421, 2020/0237903, 2020/0407697, 2019/0156911, and U.S. Pat. No. 10,940,171, each of which is specifically incorporated herein by reference in their entireties and can be used to facilitate design of the disclosed nucleic acid assemblies, including, but not limited to, the nucleic acid scaffold strand, the nucleic acid hybrid strand, the size and shape of an optional higher order structure, and other components such as additional scaffolds and staple strands, optionally accessory and functionalized molecules.

1. Nucleic Acid Scaffold

a. Nucleic Acid-Chromophore

i. Nucleic Acid Strand

The nucleic-acid chromophore includes a nucleic acid strand, also referred to as a nucleic acid scaffold strand, that hybridizes with the nucleic acid hybrid(s), also referred to as the nucleic acid hybrid strand(s), to manipulate the proximity of the two chromophores to each other.

The sequence can be a designed or desired sequence, or can be random, provided that the hybrid sequence can be designed to hybridize thereto.

In some forms, particularly where a higher order structure is desired, one or more known nucleic acid sequences are used as a scaffold sequence. In some forms the scaffold sequence is a sequence or a subset of a sequence corresponding to a bacteriophage. An exemplary scaffold sequence is a segment of the bacteriophage M13pm18. The M13mp18 single-stranded nucleic acid sequence is published as Genbank Accession Nos. X02513, M77815, and M11454.

In other forms, a sequence is randomly generated. In further forms, the template sequence for a single-stranded DNA scaffold is determined based on the required scaffold length, for example, as determined by the Eulerian circuit corresponding to the desired shape according to the described methods. If the desired sequence is longer than the input sequence template, a sequence can be randomly generated. For example, if the default template sequence is M13pm18, and the required sequence is longer than 7,249 nucleotides, a random single-stranded scaffold template sequence is generated, for example, by a computer.

In some forms, the nucleic acid scaffold sequence is between 150 to 15,000 bases in length.

When DNA is used to create dsDNA helices within a nanostructure, DNA double-stranded helices having a particular conformation can be employed. For example, double-stranded DNA can be A-form DNA, B-form DNA or Z-form DNA.

The design of the scaffold strand may include identifying the route of the scaffold nucleic acid throughout a desired structure, for example, based on the information provided in the corresponding node-edge network of the corresponding polyhedron. For example, the nodes and lines of the network correspond to the vertices and edges of the desired polyhedron. For example, Prim's formula can be used to find a breadth-first search spanning tree, one with the most branches. The spanning tree formula does not impose restrictions on the topology of the network. Therefore, the methods provide routing information for any arrangement of nodes and edges using a spanning tree to define the placement of scaffold crossovers.

ii. Chromophores

Two or more chromophores can be covalently incorporated in the backbone of the nucleic acid strand. The chromophores incorporated in the nucleic acid are adjacent to each other, forming a chromophore oligomer (also referred to herein as “oligomer”). The chromophores in the oligomer can be arranged in a linear chain or a closed ring, in which each chromophore can bind to its neighboring chromophore via a covalent bond or a non-covalent interaction, such as hydrogen bond, ionic bond, van der Waals interaction, or hydrophobic bond, or a combination thereof. In some forms, the adjacent chromophores are bound to each other via a covalent bond. The chromophores in the oligomer can be the same or different.

Exemplary chromophores that can be incorporated in the nucleic acid include, but are not limited to, a xanthene derivatives such as fluorescein, rhodamine, oregon green, eosin, and Texas red; cyanine and cyanine derivatives such as indocarbocyanine, oxacarbocyanine, thiacarbocyanine, merocyanine, and pseudoisocyanine; squaraine and squaraine derivatives, such as Seta, SeTau, and Square dyes; pentacene and pentacene derivatives; perylene diimide and perylene diimide derivatives; naphthalene and naphthalene derivatives, such as dansyl and prodan derivative; coumarin and coumarin derivatives; oxadiazole derivatives, such as pyridyloxazole, nitrobenzoxadiazole, and benzoxadiazole; anthracene derivatives, such as anthraquinones including DRAQS, DRAQ7, and CyTRAK Orange; pyrene derivatives, such as cascade blue; oxazine derivatives, such as Nile red, Nile blue, cresyl violet, and oxazine 170; acridine derivatives, such as proflavin, acridine orange, and acridine yellow; and arylmethine derivatives, such as auramine, crystal violet, and malachite green; tetrapyrrole derivatives, such as porphin, phthalocyanine, and bilirubin.

Additional examples of chromophores that can be incorporated in the nucleic acid include, but are not limited to, a CF dye (Biotium); DRAQ or CyTRAK probes (BioStatus); BODIPY (Invitrogen); Alexa Fluor (Invitrogen); DyLight Fluor (Thermo Scientific, Pierce); Atto and Tracy (Sigma-Aldrich); Fluo Probes (Interchim); Abberior Dyes (Abberior); DY and MegaStokes Dyes (Dyomics); Sulfo Cy dyes (Cyandye); HiLyte Fluor (AnaSpec); Seta, SeTau, and Square Dyes (SETA BioMedicals); Quasar and Cal Fluor dyes (Biosearch Technologies); SureLight Dyes (APC, RPEPerCP, Phycobilisomes) (Columbia Biosciences); APC, APCXL, RPE, BPE (Phyco-Biotech, Greensea, Prozyme, Flogen); or Vio Dyes (Miltenyi Biotec).

In some forms, the chromophores in the oligomer can be independently a cyanine or cyanine derivative (e.g. an indocarbocyanine (Cy3), an indodicarbocyanine (Cy5), an indotricarbocyanine (Cy7), an oxacarbocyanine, a thiacarbocyanine, a merocyanine, a pseudoisocyanine, etc.), a squaraine or squaraine derivative, a pentacene or pentacene derivative, or a perylene diimide or perylene diimide derivative. Examples of cyanine, squaraine, pentacene, perylene, and perylene diimide, and derivatives thereof are described in, for example, U.S. Pat. Nos. 10,392,659, 9,435,796, Llina, et al., Bioconjug. Chem., 31(2):194-213 (2020), US Patent Application Publication No. 2019/0270889, U.S. Pat. Nos. 6,417,402, 4,830,786, Kunzmann, et al., Nanoscale, 10:8515-8525 (2018), Tykwinski, Acc. Chem. Res., 52(8):2056-2069 (2019), Catti, J. Am. Chem. Soc., 143:9361-9367 (2021), and JP Patent Application Publication No. 2013502485.

In some forms, the chromophores in the oligomer can be independently Cy3, Cy3.5, Cy5, Cy5.5, Cy7, Cy7.5, sulfo-Cy3, sulfo-Cy5, sulfo-Cy7, Cy3 phosphoramidite, Cy3.5 phosphoramidite, Cy5 phosphoramidite, and Cy5.5 phosphoramidite; Seta, SeTau, Square dyes, dyenamo transparent green, dyenamo mareel blue, 2,4-bis[4-(N,N-dibenzylamino)-2,6-dihydroxyphenyl]squaraine, 6,13-diphenylpentacene, 6,13-bis(triisopropylsilylethynyl)pentacene, Rylene dye, pigment violet 29, pigment red 149, pigment red 179, perylene black 31, and nPDI.

In some forms, one or more of the chromophores can be a cyanine or a cyanine derivative, such as those described above. When two or more cyanine or cyanine derivatives are included in the oligomer, they can be the same or different. For example, one or more of the chromophores is a first cyanine or cyanine derivative and one or more of the chromophores is a second cyanine or cyanine derivative that is different from the first cyanine.

In some forms, each chromophore in the oligomer can be a cyanine or a cyanine derivative, such as those described above, where the cyanine or cyanine derivative can have the same or different structures. In some forms, each chromophore can be a cyanine or a cyanine derivative, such as those described above, having the same structure.

In some forms, each chromophore can bind to its neighboring chromophore via a covalent bond, forming a linear oligomer or a cyclic oligomer. In some forms, each chromophore can bind to its neighboring chromophore via a covalent bond, forming a linear oligomer. The two or more chromophores can be incorporated in the backbone of the nucleic acid strand using a variety of chemical reactions known in the art, such as phosphoramidite chemistry, H-phosphonate chemistry, click reactions, Vorbruggen nucleosidation reaction, Heck reaction, chemoenzymatic translycosylation methods, or Pd-assisted cross-coupling reactions, see for example, those described in Luke, et al., Chem. Soc. Rev., 50:5126-5164 (2021).

In some forms, the oligomer incorporated in the backbone of the nucleic acid can have the structure of Formula I.

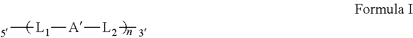

where each occurrence of A′ is a chromophore, such as any of the chromophores described above; each occurrence of L₁ and L₂ is independently absent, a bond (a single bond or a double bond), oxygen, sulfur, amino, amido, azido, carbonyl, ether, polyether, sulfide, sulfonyl, sulfonic acid, phosphoryl, phosphonyl, phosphate, thiol, substituted or unsubstituted alkyl, substituted or unsubstituted alkenyl, substituted or unsubstituted aryl, substituted or unsubstituted polyaryl, substituted or unsubstituted heteroaryl, substituted or unsubstituted polyheteroaryl, substituted or unsubstituted heterocyclyl, substituted or unsubstituted cycloalkyl, or substituted or unsubstituted cycloalkenyl; the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol, or a combination thereof; n can be a positive integer ≥2, such as from 2 to 100, from 2 to 80, from 2 to 60, from 2 to 50, from 2 to 40, from 2 to 30, from 2 to 20, from 2 to 10, from 2 to 8, from 2 to 6, from 2 to 5, from 2 to 4, or 2 or 3; and a first terminal chromophore is bound to the 5′-end of its neighboring nucleic acid and a second terminal chromophore is bound to the 3′-end of its neighboring nucleic acid.

In some forms of Formula I, the substituents can be independently an unsubstituted alkyl, an unsubstituted alkenyl, a substituted or unsubstituted heterocyclyl, a substituted or unsubstituted aryl, a substituted or unsubstituted polyaryl, an oxo, an alkoxyl, a halogen, a hydroxyl, a haloalkyl, or an amino, or a combination thereof. In some forms of Formula I, the substituents can be independently an unsubstituted alkyl (e.g., methyl, ethyl, propyl, butyl, pentyl, hexyl, etc.), an unsubstituted alkenyl (methylene, ethylene, propylene, butylene, pentylene, hexylene, etc.), an unsubstituted heterocyclyl, a phenyl, an unsubstituted polyaryl, an alkoxyl (e.g. —O-alkyl), a halogen, a hydroxyl, or a haloalkyl, or a combination thereof.

In some forms, the two or more chromophores can be incorporated in the backbone of the nucleic acid strand using phosphoramidite chemistry, such that each chromophore is bound to the neighboring chromophore via a phosphate group and each terminal chromophore is bound to a neighboring nucleic acid via a phosphate group. In some forms, the oligomer incorporated in the backbone of the nucleic acid strand can have the structure of Formula II.

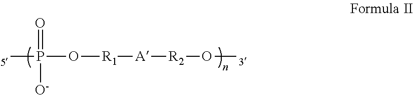

wherein A′ can be a chromophore, such as any of the chromophores described above; R₁ and R₂ can be independently absent or a substituted or unsubstituted alkyl group; n can be a positive integer ≥2, such as from 2 to 100, from 2 to 80, from 2 to 60, from 2 to 50, from 2 to 40, from 2 to 30, from 2 to 20, from 2 to 10, from 2 to 8, from 2 to 6, from 2 to 5, from 2 to 4, or 2 or 3; and a first terminal chromophore is bound to the 5′-end of its neighboring nucleic acid and a second terminal chromophore is bound to the 3′-end of its neighboring nucleic acid.

In some forms of Formula II, R₁ and R₂ can be independently absent, a substituted or unsubstituted C₁-C₂₀ alkyl group, a substituted or unsubstituted C₁-C₁₅ alkyl group, a substituted or unsubstituted C₁-C₁₀ alkyl group, a substituted or unsubstituted C₁-C₈ alkyl group, a substituted or unsubstituted C₁-C₆ alkyl group, a substituted or unsubstituted C₁-C₄ alkyl group, an unsubstituted C₁-C₂₀ alkyl group, an unsubstituted C₁-C₁₅ alkyl group, an unsubstituted C₁-C₁₀ alkyl group, an unsubstituted C₁-C₈ alkyl group, an unsubstituted C₁-C₆ alkyl group, an unsubstituted C₁-C₄ alkyl group, such as a methyl, ethyl, propyl, butyl, pentyl, hexyl, etc.

In some forms of Formulae I and II, n can be 2 or 3 (a dimer or trimer). In some forms of Formulae I and II, n can be 2. In some forms of Formulae I and II, n can be 3. In some forms of Formulae I and II, each occurrence of A′ can be a cyanine, a squaraine, a pentacene, or a perylene diimide, or a derivative thereof, such as those described above. In some forms of Formulae I and II, one or more A′ can be a cyanine or a cyanine derivative described above, such as Cy3, Cy3.5, Cy5, Cy5.5, Cy7, Cy7.5, sulfo-Cy3, sulfo-Cy5, sulfo-Cy7, etc. In some forms of Formulae I and II, each occurrence of A′ can be a cyanine or a cyanine derivative described above, such as Cy3, Cy3.5, Cy5, Cy5.5, Cy7, Cy7.5, sulfo-Cy3, sulfo-Cy5, sulfo-Cy7, etc. In some forms of Formulae I and II, A′ can have the structure of:

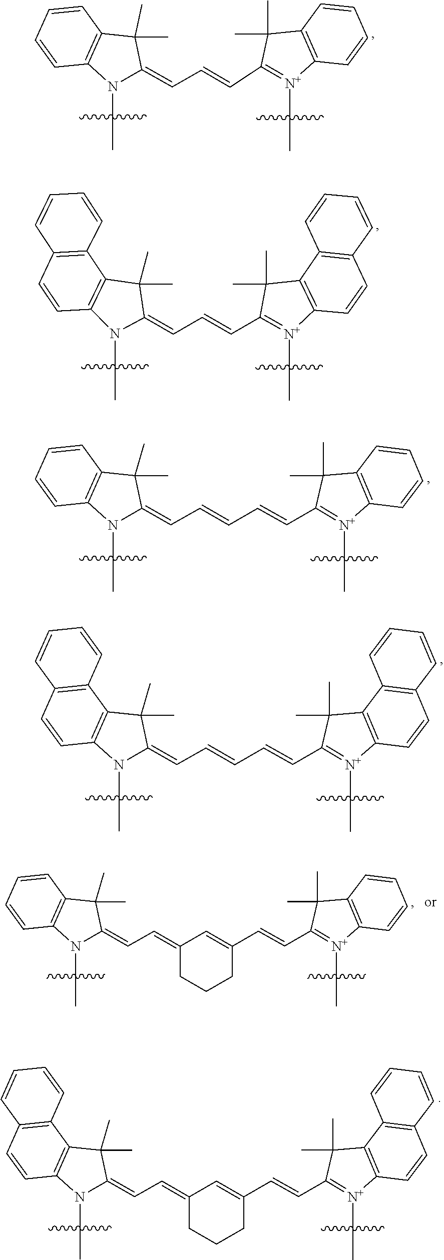 [SMILES: *N1/C(=C/C=C/C2=[N+](*)c3ccccc3C2(C)C)C(C)(C)c2ccccc21 ; *N1/C(=C/C=C/C2=[N+](*)c3ccc4ccccc4c3C2(C)C)C(C)(C)c2c1ccc1ccccc21 ; *N1/C(=C/C=C/C=C/C2=[N+](*)c3ccccc3C2(C)C)C(C)(C)c2ccccc21 ; *N1/C(=C/C=C/C=C/C2=[N+](*)c3ccc4ccccc4c3C2(C)C)C(C)(C)c2c1ccc1ccccc21 ; *N1/C(=C/C=C2C=C(/C=C/C3=[N+](*)c4ccccc4C3(C)C)CCC/2)C(C)(C)c2ccccc21 ; *N1/C(=C/C=C2C=C(/C=C/C3=[N+](*)c4ccc5ccccc5c4C3(C)C)CCC/2)C(C)(C)c2c1ccc1ccccc21]

In some forms of Formula II, the chromophore A′ is indocarbocyanine, having a core structure of

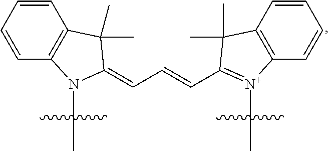 [SMILES: *N1/C(=C/C=C/C2=[N+](*)c3ccccc3C2(C)C)C(C)(C)c2ccccc21]

and R₁ and R₂ can be independently a substituted or unsubstituted C₁-C₂₀ alkyl group, a substituted or unsubstituted C₁-C₁₅ alkyl group, a substituted or unsubstituted C₁-C₁₀ alkyl group, a substituted or unsubstituted C₁-C₈ alkyl group, a substituted or unsubstituted C₁-C₆ alkyl group, a substituted or unsubstituted C₁-C₄ alkyl group, an unsubstituted C₁-C₂₀ alkyl group, an unsubstituted C₁-C₁₅ alkyl group, an unsubstituted C₁-C₁₀ alkyl group, an unsubstituted C₁-C₈ alkyl group, an unsubstituted C₁-C₆ alkyl group, an unsubstituted C₁-C₄ alkyl group, such as a methyl, ethyl, propyl, butyl, pentyl, hexyl, etc.

In some forms of Formula II, the chromophore A′ is indodicarbocyanine, having a core structure of

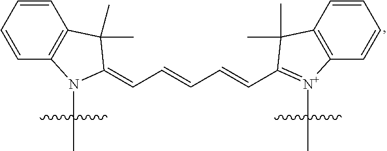 [SMILES: *N1/C(=C/C=C/C=C/C2=[N+](*)c3ccccc3C2(C)C)C(C)(C)c2ccccc21]

and R₁ and R₂ can be independently a substituted or unsubstituted C₁-C₂₀ alkyl group, a substituted or unsubstituted C₁-C₁₅ alkyl group, a substituted or unsubstituted C₁-C₁₀ alkyl group, a substituted or unsubstituted C₁-C₈ alkyl group, a substituted or unsubstituted C₁-C₆ alkyl group, a substituted or unsubstituted C₁-C₄ alkyl group, an unsubstituted C₁-C₂₀ alkyl group, an unsubstituted C₁-Cis alkyl group, an unsubstituted C₁-C₁₀ alkyl group, an unsubstituted C₁-C₈ alkyl group, an unsubstituted C₁-C₆ alkyl group, an unsubstituted C₁-C₄ alkyl group, such as a methyl, ethyl, propyl, butyl, pentyl, hexyl, etc.

In some forms, the oligomer incorporated in the backbone of the nucleic acid strand can have the structure of Formula III.

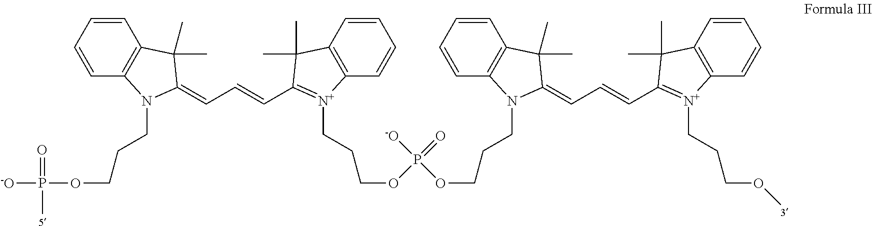

b. Nucleic Acid Hybrid

The disclosed structures typically include a nucleic acid hybrid strand that hybridizes to the nucleic acid of the scaffold and controls to the proximity of two or more chromophores of the scaffold strand. The hybrid strand can be of any length. In some forms, the nucleic acid scaffold and hybrid strand form e.g., a double duplex. In some forms, the scaffold and hybrid strand can for structure of define, e.g., higher order shape. Forming a desired shape can be accomplished by folding a long single stranded polynucleotide scaffold strand into a desired shape or structure using a number of small “staple strands” as glue to hold the scaffold in place. Thus, in some form, the hybrid strand is a staple strand.

Typically, the number of staple strands will depend upon the size of the scaffold strand, the complexity of the shape or structure, the types of crossover motifs employed, and the number of helices per edge. For example, for relatively short scaffold strands (e.g., about 150 to 1,500 base in length) and/or simple structures the number of staple strands are small (e.g., about 5, 10, 50 or more). For longer scaffold strands (e.g., greater than 1,500 bases) and/or more complex structures, the number of staple strands can be several hundreds to thousands (e.g., 50, 100, 300, 600, 1,000 or more helper strands). Using parallel crossover motifs, however, the number of staples can be reduced, even to zero. The choice of staple strands and, in some instances, the programmed self-hybridization of the scaffold strand, determine the pattern. In some forms, a software program is used to identify the staple strands needed to form a given design.

Staples can be on vertices, edges with scaffold crossovers, and edges without scaffold crossovers. Preferably, the hybrid strand forms part of a crossover.

c. Composition of the Nucleic Acids

Nucleic acid scaffold and hybrid strands of the disclosed compositions can be composed of deoxyribonucleic acids, ribonucleic acids, locked nucleic acids (LNA), peptide nucleic acids (PNA), etc. The nucleic acids can be formed of deoxyribonucleotides (DNA), ribonucleotides (RNA), or analogs or modified nucleotides thereof. When modified nucleotides are incorporated into nucleic acid scaffold strand or hybrid, the modified nucleotides can be incorporated as a percentage or ratio of the total nucleotides used in the preparation of the nucleic acids. In some forms, the modified nucleotides represent 0.1% or more than 0.1% of the total number of nucleotides in the sequence, up to or approaching 100% of the total nucleotides present. For example, the relative amount of modified nucleotides can be between 0.1% and 100% inclusive, such as 0.1%-0.5%, 1%-2%, 1%-5%, 1%-10%, 10%-20%, 20%-30%, 30%-40%, 40%-50%, or more than 50% of the total, up to and including 100%, such as 60%, 70%, 75%, 80%, 85%, 90%, 95% or 99% of the total.

In a certain form, a sequence of nucleic acids includes a single modified nucleotide, or two, or three modified nucleotides. In some forms, nucleic acid nanostructures contain one, or more than one, up to 100 modified nucleotides or more in every edge. In other forms, the number of modified nucleotides correlates with the size of the nanostructure, or the shape, or the number of faces or edges, or vertices of the nanostructure. For example, in some forms, nucleic acid nanostructures include the same or different numbers of modified nucleotides within every edge or vertex. In some forms, the modified nucleotides are present at the equivalent position in every structurally equivalent edge of the nanostructure. In some forms, nucleic acid nanostructures include modified nucleotides at precise locations and in specific numbers or proportions as determined by the design process. Therefore, in some forms, nucleic acid assemblies include a defined number or percentage of modified nucleotides at specified positions within the structure. In some forms, nucleic acid nanostructures produced according to the described methods include more than a single type of modified nucleic acid. In exemplary forms, nucleic acid nanostructures include one type of modified nucleic acid on every edge, or mixtures of two or more different modified nucleic acids on every edge. Therefore, when a single type of modified nucleic acid is present at an edge of the structure, each edge can include a different type of modification relative to every other edge.

Examples of modified nucleotides that can be included within the described nanostructures include, but are not limited to, diaminopurine, S²T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

In some forms phosphorothioate modified backbone on the DNA nucleotide staples or on the scaffold is used to improve stability of the DNA nanostructures to degradation by exonuclease. For example, in some forms the nucleic acid nanostructures include modified nucleic acids that protect one or more regions of the nanostructure from enzymic degradation or disruption in vivo. In some forms, nucleic acid nanostructures include modified nucleic acids at specific locations within the structure that direct the timing of the enzymic degradation of specific parts of the structure. For example, modifications can be designed to prevent degradation, or to enhance the likelihood of degradation of one or more edges before or after different edges within the same structure. In this way, modifications that enhance or reduce protection or enzymic degradation of one or more parts of a nanostructure in vivo can drive or facilitate structural changes in the structure, for example, for example to enhance or alter the half-life of a given structure in vivo.

Locked nucleic acid (LNA) is a family of conformationally locked nucleotide analogues which, amongst other benefits, imposes truly unprecedented affinity and very high nuclease resistance to DNA and RNA oligonucleotides (Wahlestedt C, et al., Proc. Natl Acad. Sci. USA, 975633-5638 (2000); Braasch, D A, et al., Chem. Biol. 81-7 (2001); Kurreck J, et al., Nucleic Acids Res. 301911-1918 (2002)). In some forms, the nucleic acids are synthetic RNA-like high affinity nucleotide analogue, locked nucleic acids. In some forms, the scaffolded DNAs are locked nucleic acids. In other forms, the staple strands are locked nucleic acids.

Peptide nucleic acid (PNA) is a nucleic acid analog in which the sugar phosphate backbone of natural nucleic acid has been replaced by a synthetic peptide backbone usually formed from N-(2-amino-ethyl)-glycine units, resulting in an achiral and uncharged mimic (Nielsen P E et al., Science 254, 1497-1500 (1991)). It is chemically stable and resistant to hydrolytic (enzymatic) cleavage. In some forms, the scaffolded DNAs are PNAs. In other forms, the staple strands are PNAs.

In some forms PNAs, DNAs, RNAs, or LNAs are used for capture, or proteins or other small molecules of interest to target, or otherwise interact with complementary binding sites on structured RNAs, or DNAs. In other forms, a combination of PNAs, DNAs, RNAs and/or LNAs is used in the formation of structured nucleic acid nanostructures.

In some forms, the compositions include a combination of PNAs, DNAs, and/or LNAs. In some forms, a combination of PNAs, DNAs, and/or LNAs is used for the staple strands.

In some forms, nucleic acid nanostructures include one or more nucleic acids conjugated to polymers. Exemplary polymers that can be conjugated to nucleic acids include biodegradable polymers, non-biodegradeable polymers, cationic polymers and dendrimers. For example, a non-limiting list of polymers that can be coupled to nucleic acids within the nucleic acid nanostructures includes poly(beta-amino esters); aliphatic polyesters; polyphosphoesters; poly(L-lysine) containing disulfide linkages; poly(ethylenimine) (PEI); disulfide-containing polymers such as DTSP or DTBP crosslinked PEI; PEGylated PEI crosslinked with DTSP; Crosslinked PEI with DSP; Linear SS-PEI; DTSP-Crosslinked linear PEI; branched poly(ethylenimine sulfide) (b-PEIS). Typically, the polymer has a molecular weight of between 500 Da and 20,000 Da, inclusive, for example, approximately 1,000 Da to 10,000 Da, inclusive. In some forms, the polymer is ethylene glycol. In some forms, the polymer is polyethylene glycol. In an exemplary form, one or more polymer are conjugated to the nucleic acids within one or more of the staples. Therefore, in some forms, one or more types of polymers conjugated to staple strands are used to coat the nucleic acid nanostructure with the one or more polymers. In some forms, one or more types of polymers conjugated to nucleic acids in the scaffold sequence are used to coat the used to coat the DNA nucleic acid nanostructure with the one or more polymers.

2. Exemplary Nucleic Acid Structures

The nucleic acid scaffold and hybrid strand typically hybridize in a manner that controls the distance between two chromophores of the scaffold strand. By changing the length and/or sequence of the hybrid strand, the practitioner can increase or decrease the distance between the chromophores and thus manipulate their activity. In some forms, the scaffold strand and hybrid strand interact in the context of a double crossover.

a. Double Crossovers Tile

In the most preferred forms, the nucleic acid scaffold and hybrid strand interact as part of a double crossover tile. The double crossover can be antiparallel or parallel.

i. Antiparallel Crossovers

In some forms, the nucleic acid scaffold and hybrid strands hybridize through an antiparallel double crossover. The terms “DX crossover,” or “antiparallel crossover,” or “DX motif” are used interchangeably, and refer to an antiparallel double crossover nucleic acid motif including two four-arm Holliday junctions, joined by two helical arms at two adjacent arms. The antiparallel orientation of the nucleic acid helical domains in antiparallel DX motifs implies that the major grooves of one nucleic acid helix faces the minor groove of the other engaged helices come together in each turn.

ii. Parallel Crossovers

The term “PX crossover,” or “parallel crossover,” or “PX motif” are used interchangeably to refer to a four-stranded DNA motif wherein two parallel double helices are joined by reciprocal exchange (crossing over) of strands of the same polarity at every point where the strands come together (see Seeman, Nano Letters 1, (1), pp. 22-26 (2001); Wang, PNAS, V. 107 (28), pp. 12547-12552 (2010)). No strand breakage and rejoining is needed, because two double helices can form PX-DNA merely by inter-wrapping. The reciprocal exchange between two double stranded nucleic acid helices can occur between two helices having either the same or opposite stand polarity. An exemplary PX motif is the “paranemic crossover.” PX motifs are usually followed by a pair of numbers, e.g. PX65 motif, that describe the number of base pairs in the major groove and minor groove of the double helices, respectively, between parallel crossovers. The number of base pairs in the major groove is typically greater than that in the minor groove. Exemplary, PX motifs include PX65, PX75, PX85, PX95, PX64, PX74, and PX66 (Maiti, et al., Biophysical Journal. 90, 1463-1479 (2006); Shen, et al., J. Am. Chem. Soc. 126, 1666-1674 (2004)).

b. Exemplary Structures

As introduced above, the nucleic acid scaffold and hybrid strands can hybridize in the context of a large nucleic acid structure. In some forms, nucleic acid scaffold and hybrid strands hybridize to form a crossover, which can be antiparallel or parallel. In some forms, this crossover is just one or several crossovers. Thus, the hybrid strand can form a crossover tile (i.e., DX or PX tile), which can be just one of series of tiles in a large structure. The structure can be a 2D structure of a 3D structure.

Exemplary shapes include three-dimensional structures, including, but not limited to, Platonic polyhedrons, Archimedean polyhedrons, Johnson polyhedrons, Catalan solids, or asymmetric three-dimensional structures. In some forms, the structure has a programmed geometry that is topologically equivalent to that of a sphere. In other forms, the structure has a programmed geometry that is topologically distinct to that of a sphere. For example, the structures can be nested structures, and toroidal structures. In other forms, the structure has a programmed geometry that is topologically equivalent to a plane. For example, structures include triangular mesh, square mesh, or other mesh.

Structures can be selected based upon one or more design criteria, or can be selected randomly. In some forms, structures are selected based on existing ‘natural’ 3-dimensional organizations (e.g., virus capsids, antigens, toxins, etc.). Therefore, in some forms, shapes are designed for use directly or as part of a system to mediate a biological or other responses which are dependent upon, or otherwise influenced by 3D geometric spatial properties. For example, in some forms, all or part of a structure is designed to include architectural features known to elicit or control one or more biological functions. In some forms, structures are designed to fulfill the 3D geometric spatial requirements to induce, prevent, stimulate, activate, reduce or otherwise control one or more biological functions. Typically, the desired shape defines a specific geometric form that will constrain the other physical parameters, such as the absolute size of the particle. For example, the minimum size of nucleic acid nanostructures designed according to the described methods will depend upon the degree of complexity of the desired shape.

In some forms, nucleic acid nanostructures are designed using methods that determine the single-stranded oligonucleotide staple sequences that can be combined with the target sequence to form a complete three-dimensional nucleic acid nanostructure of a desired form and size. Therefore, in some forms, the methods include the automated custom design of nucleic acid nanostructure corresponding to a target nucleic acid sequence. For example, in some forms, a robust computational approach is used to generate DNA-based wireframe polyhedral structures of arbitrary scaffold sequence, symmetry and size. In particular forms, design of a nanostructure corresponding to the target nucleic acid sequence, includes providing information as geometric parameters corresponding to the desired form and dimensions of the nanostructure, which are used to generate the sequences of oligonucleotide “staples” that can hybridize to the target nucleic acid “scaffold” sequence to form the desired shape. Typically, the target nucleic acid is routed throughout the Eulerian circuit of the network defined by the wire-frame geometry of the nanostructure.

A step-wise, top-down approach has been proven for generating DNA nanostructure origami objects of any regular or irregular wireframe polyhedron, with edges composed of a multiple of two number of helices (i.e., 2, 4, 6, etc.) and with edge lengths a multiple of 10.5 rounded down to the closest integer. Exemplary methods for the top-down design of nucleic acid nanostructures of arbitrary geometry are described in Venziano et al, Science, 352:6293 (2016), Jun et al., ACS Nano, 2019, 10.1021/acsnano.8b08671, WO2017089567A1, and WO2017089570A1, the contents of which are incorporated by reference in their entireties.

In other forms, the sequence of the nanostructure is designed manually, or using alternative computational sequence design procedures. Exemplary design strategies that can be incorporated into the methods for making and using NMOs include single-stranded tile-based DNA origami (Ke Y, et al., Science 2012); brick-like DNA origami, for example, including a single-stranded scaffold with helper strands (Rothemund, et al., and Douglas, et al.); and purely single-stranded DNA that folds onto itself in PX-origami, for example, using paranemic crossovers.

Alternative structured NMOs include bricks, bricks with holes or cavities, assembled using DNA duplexes packed on square or honeycomb lattices (Douglas et al., Nature 459, 414-418 (2009); Ke Y et al., Science 338: 1177 (2012)). Paranemic-crossover (PX)-origami in which the nanostructure is formed by folding a single long scaffold strand onto itself can alternatively be used, provided bait sequences are still included in a site-specific manner. Further diversity can be introduced such as using different edge types, including 6-, 8-, 10, or 12-helix bundle. Further topology such as ring structure is also useable for example a 6-helix bundle ring.

i. 2-Dimensional Wireframe Structures

Structures can be any solid in two dimensions. Therefore, structures can be a grid or mesh or wireframe topologically similar to a 2D surface or plane. The grid or mesh can be composed of regular or irregular geometries that can be tessellated over a surface.

Exemplary structures include triangular lattices, square lattices, pentagonal lattices, or lattices of more than 5 sides. 2D structures can be designed to have varied length and thickness in each dimension. In some forms, the edges of 2D nanostructures include a single nucleic acid helix. In other forms, the edges of 2D nanostructures include two or more nucleic acid helices. For example, in some forms, each edge of the 2D nanostructure includes 2 helices, 4 helices, 6 helices or 8 helices, or more than 8 helices, up to 100 helices per edge, although theoretically unlimited in number.

ii. Polyhedral Structures

Structures can be any solid in three dimensions that can be rendered with flat polygonal faces, straight edges and sharp corners or vertices.

Exemplary basic structures include cuboidal structures, icosahedral structures, tetrahedral structures, cuboctahedral structures, octahedral structures, and hexahedral structures. In some forms, the structure is a convex polyhedron, or a concave polyhedron. For example, in some forms, the structure is a uniform polyhedron that has regular polygons as faces and is isogonal. In other forms, the structure is an irregular polyhedron that has unequal polygons as faces. In further forms, the structure is a truncated polyhedral structure, such as truncated cuboctahedron.

Platonic polyhedrons include polyhedrons with multiple faces, for example, 4 faces (tetrahedron, (1)), 6 faces (cube or hexahedron (2), 8 faces (octahedron), 12 faces (dodecahedron), and 20 faces (icosahedron).

In some forms, the structure is a nucleic acid assembly that has a non-spherical geometry. Therefore, in some forms, the structure has a geometry with “holes.” Exemplary non-spherical geometries include toroidal polyhedra and nested shapes. Exemplary toroidal polyhedra include a torus and double torus. Exemplary topologies of nested shapes include nested cube and nested octahedron.

In other forms, target structures can be a combination of one or more of the same or different polyhedral forms, linked by a common contiguous edge.

iii. Reinforced Polyhedral Structures

In some forms, the structure is a reinforced structure. Reinforced structures are structures that share the same polyhedral form as the equivalent, non-reinforced structure, and include one or more additional edges spanning between two vertices. Typically, the reinforced structure contains at least one or more edges than the corresponding non-reinforced structure. In some forms, additional structural elements that appear as “cross-bars” spanning between two vertices are introduced.

In some forms, a structure is reinforced by the addition of one or more edges passing internally within the space enclosed by the structure. Therefore, in some forms reinforced structures encapsulate a smaller volume than the corresponding non-reinforced structure. In other forms, a structure is reinforced by the addition of one or more edges that connects vertices by spanning a face of the polyhedron. In further forms, a polyhedral nanostructure is reinforced by including one or more additional edges that connect vertices by spanning a face of the polyhedron and one or more additional edges that connect vertices by passing internally within the space enclosed by the structure. In some forms, a polyhedral nanostructure is reinforced by addition of one or more edges that bisects a face of the polyhedron and addition of one or more vertices.

iv. Other Structures

In some forms the desired structure has a shape that is visually or geometrically similar to a biological structure, such as the shape of a viral particle, or a sub-component of a viral particle; a protein; or a sub-component of a protein.

3. Assembling the Nucleic Acid Assembly

Typically, annealing can be carried out according to the specific parameters of the nucleic acid hybrid and scaffold and any further staple sequences. For example, the oligonucleotide hybrid and optional staples are mixed in the appropriate quantities in an appropriate reaction volume. In preferred forms, the hybrid and optional staple strand mixes are added in an amount effective to maximize the yield and correct assembly of the nanostructure. For example, in some forms, the hybrid and optional staple strand mixes are added in molar excess of the scaffold strand. In an exemplary form, the staple strand mixes are added at a 10-20× molar excess of the scaffold strand. In some forms, the synthesized oligonucleotides hybrid and optional staples are mixed with the scaffold strand and annealed by slowly lowering the temperature (annealing) over the course of 1 to 48 hours.

Material usage for assembly can be minimized and assembly hastened by use of microfluidic automated assembly devices. For example, in certain forms, the oligonucleotide hybrid and optional staples are added in one inlet, the scaffold can be added in a second inlet, with the solution being mixed using methods known in the art, and the mix traveling through an annealing chamber, wherein the temperature steadily decreases over time or distance. The output port then contains the assembled nanostructure for further purification or storage. Similar strategies can be used based on digital droplet-based microfluidics on surfaces to mix and anneal solutions and applied to purely single-stranded oligo-based nanostructures or single-stranded scaffold origami in the absence of helper strands.

B. Encapsulation

In some forms, the nucleic acid assemblies are encapsulated. Suitable encapsulating agents include gel-based beads, protein viral packages, micelles, mineralized structures, siliconized structures, or polymer packaging.

In some forms, the encapsulating agents are viral capsids or a functional part, derivative and/or analogue thereof. In some forms, the NSOs are viral like particles, with nucleic acid content enveloped by protein content on the surface. Viral capsids can be derived from retroviruses, human papilloma viruses, M13 viruses, adeno viruses adeno-associated viruses, for example, adenovirus 16. In preferred forms, viral capsids used for encapsulating NSOs do not interfere with the overhang tags i.e. overhang tags are accessible for purification purposes.

In some forms, the encapsulating agents are lipids forming micelles, or liposomes surrounding the nucleic acid. In some forms, micelles, or liposomes are formed from one or more lipids, which can be neutral, anionic, or cationic at physiologic pH. Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including, but not limited to, 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

Suitable cationic lipids in the micelles, or the liposomes include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N—(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N,N,N′,N′-tetramethyl-, N′-bis(2-hydroxylethyl)-2,3-dioleoyloxy-1,4-butanediammonium iodide. In one form, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one form, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids may be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine).

In some forms, the encapsulating agents are natural or synthetic polymers. Representative natural polymers are proteins, such as zein, serum albumin, gelatin, collagen, and polysaccharides, such as cellulose, dextrans, and alginic acid. Representative synthetic polymers include polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, alkyl cellulose, hydroxyalkyl celluloses, cellulose ethers, cellulose esters, nitrocelluloses, polymers of acrylic and methacrylic esters, poly[lactide-co-glycolide], polyanhydrides, polyorthoestersblends and copolymers thereof. Specific examples of these polymers include cellulose acetate, cellulose propionate, cellulose acetate butyrate, cellulose acetate phthalate, carboxymethyl cellulose, cellulose triacetate, cellulose sulphate, poly(methyl methacrylate), (poly(ethyl methacrylate), poly(butyl methacrylate), Poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate), poly(vinyl chloride), polystyrene and polyvinylpyrrolidone, polyurethane, polylactides, poly(butyric acid), poly(valeric acid), poly[lactide-co-glycolide], polyanhydrides, polyorthoesters, poly(fumaric acid), and poly(maleic acid).

In some forms, the encapsulating agents are mineralized, for example, calcium phosphate mineralization of alginate beads, or polysaccharides. In other forms, the encapsulating agents are siliconized. In one form, the nucleic acid is packaged in a mineral structure.

In some forms, the encapsulating agents are metal oxide particles. Exemplary metal oxide encapsulating agents include silicon dioxide (SiO₂) and titanium dioxide (TiO₂), that can be mesoporous, compact, or structured. In some forms, the DNA is adsorbed on the surface of a modified metal oxide particle then coated with polyelectrolytes, for example poly(diallyldimethylammonium chloride), poly(acrylamide-co-diallyldimethylammonium chloride), and poly(allylamine hydrochloride).

C. Photophysical Properties

The photophysical properties of the oligomer containing two or more adjacent chromophores can be evaluated by quantum yield, energy dependent optical density, emission energetic profile, and/or excited state lifetime. Techniques for measuring these parameters are known, for example, by measuring the emission and/or excitation spectra of a nucleic acid-chromophore.

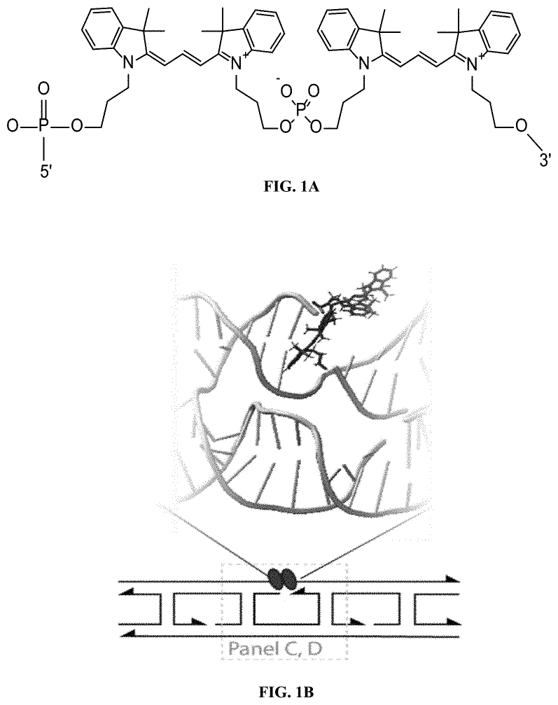

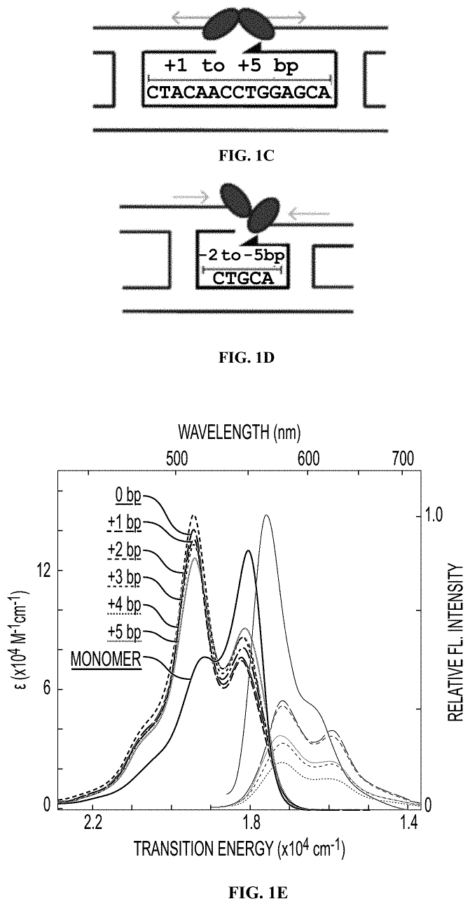

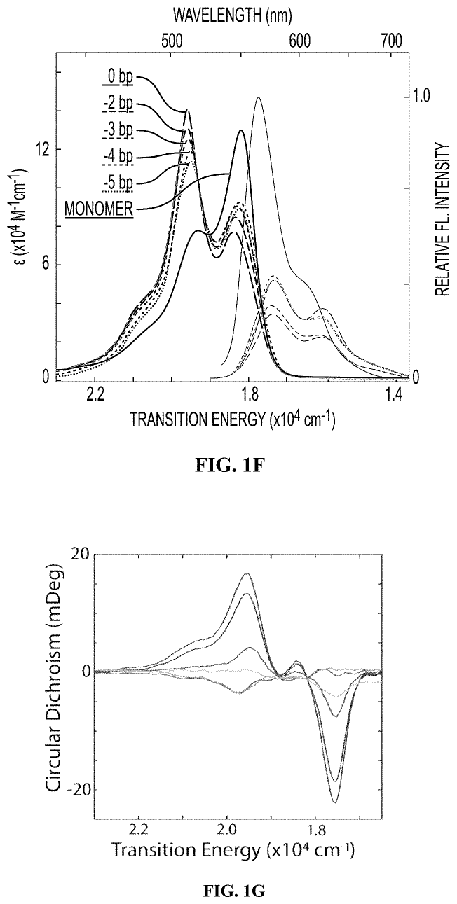

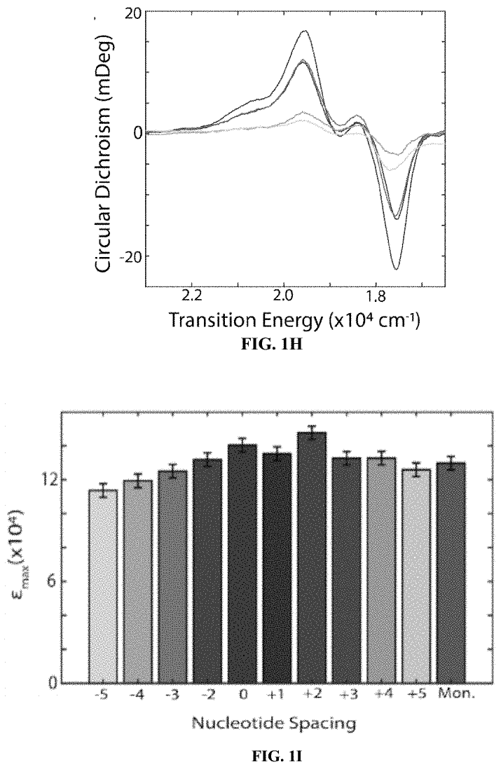

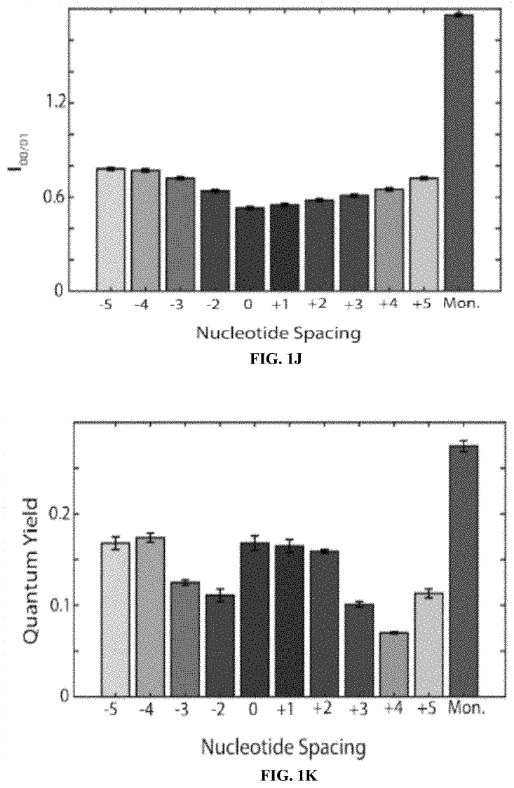

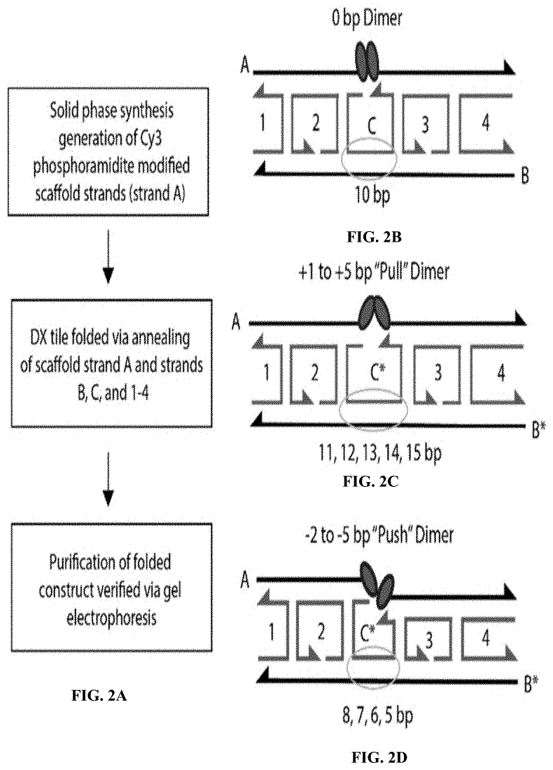

One or more of the photophysical properties of the oligomer that is incorporated in the nucleic acid strand can be altered by changing the length of the nucleic acid hybrid in the nucleic acid scaffold that is opposite the adjacent chromophores. Typically, the nucleic acid hybrid does not contain any chromophore. For example, by adding or removing one or more complementary bases in the nucleic acid hybrid, the adjacent chromophores can be pulled apart or pushed together, thereby change the electronic coupling of the oligomer and result in a change in one or more photophysical properties of the oligomer (see, e.g. FIGS. 1C-1D and FIGS. 2B-2D).

In some forms, the quantum yield of the oligomer can be changed by at least 5%, at least 10%, at least 20%, at least 30%, at least 40%, at least 50%, in a range from 10% to 80%, from 10% to 70%, from 10% to 60%, or from 10% to 50%, by adding or removing one or more complementary bases in the nucleic acid hybrid, such as by adding or removing from 1 to 10, from 1 to 9, from 1 to 8, from 1 to 7, from 1 to 6, from 1 to 5, from 1 to 4, from 1 to 3, or 1 or 2 complementary bases in the nucleic acid hybrid, compared to the quantum yield of the oligomer before adding or removing the complementary bases.

In some forms, the energy dependent optical density of the oligomer can be changed by at least 5%, at least 8%, at least 10%, in a range from 5% to 30%, from 5% to 25%, from 5% to 20%, from 5% to 25%, or from 5% to 20% by adding or removing one or more complementary bases in the nucleic acid hybrid, such as by adding or removing from 1 to 10, from 1 to 9, from 1 to 8, from 1 to 7, from 1 to 6, from 1 to 5, from 1 to 4, from 1 to 3, or 1 or 2 complementary bases in the nucleic acid hybrid, compared to the energy dependent optical density of the oligomer before adding or removing the complementary bases.

In some forms, one or more peaks of the emission energetic profile of the oligomer can be changed by at least 5%, at least 8%, at least 10%, in a range from 5% to 30%, from 5% to 25%, from 5% to 20%, from 5% to 25%, or from 5% to 20%, by adding or removing one or more complementary bases in the nucleic acid hybrid, such as by adding or removing from 1 to 10, from 1 to 9, from 1 to 8, from 1 to 7, from 1 to 6, from 1 to 5, from 1 to 4, from 1 to 3, or 1 or 2 complementary bases in the nucleic acid hybrid, compared to the same peak(s) of the emission energetic profile of the oligomer before adding or removing the complementary bases.

In some forms, the excited state lifetime of the oligomer can be changed by at least 5%, at least 10%, at least 20%, at least 30%, at least 40%, at least 50%, in a range from 10% to 80%, from 10% to 70%, from 10% to 60%, or from 10% to 50%, by adding or removing one or more complementary bases in the nucleic acid hybrid, such as by adding or removing from 1 to 10, from 1 to 9, from 1 to 8, from 1 to 7, from 1 to 6, from 1 to 5, from 1 to 4, from 1 to 3, or 1 or 2 complementary bases in the nucleic acid hybrid, compared to the excited state lifetime of the oligomer before adding or removing the complementary bases.

In some forms, each of the quantum yield, energy dependent optical density, emission energetic profile, and excited state lifetime can be changed in any of the range described above, by adding or removing one or more complementary bases in the nucleic acid hybrid, such as by adding or removing from 1 to 10, from 1 to 9, from 1 to 8, from 1 to 7, from 1 to 6, from 1 to 5, from 1 to 4, from 1 to 3, or 1 or 2 complementary bases in the nucleic acid hybrid.

In some forms, the alteration in the one or more photophysical properties of the oligomer is sufficient to distinguish a single altered nucleic acid assembly from a single unaltered nucleic acid assembly.

Additionally or alternatively to the change by adding or removing complementary bases in the nucleic acid hybrid, one or more of the photophysical properties of the oligomer that is incorporated in the nucleic acid strand can be altered by changing the local environment of the nucleic acid assembly, such as by changing the solvent polarity with which the nucleic acid assembly is in contact and/or by encapsulating the nucleic acid assembly in an organic or inorganic media (e.g. silica). For example, the excited state lifetime of the oligomer can be changed by at least 5%, at least 8%, at least 10%, in a range from 5% to 30%, from 5% to 25%, from 5% to 20%, from 5% to 25%, or from 5% to 20%, by changing the solvent polarity by adding a percentage of dimethyl formaldehyde (“DMF”) in water, such as by adding 5% to 30% DMF in water.

### III. Methods of Use

The disclosed nucleic acid-chromophores and nucleic acid assemblies can used in numerous settings and methods that can make use of the detection and measuring of emissions from the chromophores. For example, the disclosed nucleic acid-chromophores and nucleic acid assemblies in any setting or method in which labels are or can be used.

A. Detection of Change in a Nucleic Acid Assembly

Generally, the method includes (i) measuring one or more of the photophysical properties of the adjacent chromophores, wherein the change is detected if one or more of the measured photophysical properties of the adjacent chromophores is altered compared to a reference photophysical property.

The reference photophysical property can be the photophysical property of the nucleic acid assembly in the absence of a change in the nucleic acid assembly.

The change in the nucleic acid assembly can be produced by interaction of a molecule of interest with a biological or nonbiological material to which the nucleic acid assembly is operably coupled.

The change in the nucleic acid assembly can be produced by correct assembly of a scaffold origami of which the nucleic acid assembly becomes a part.

The change in the nucleic acid assembly can be produced by a change in the milieu of the nucleic acid assembly.

The change in milieu of the nucleic acid assembly can be a change in the polarity of solvent in which the nucleic acid assembly is dissolved or suspended.

B. Multiplex Detection

Generally, the method of labeling each of a first plurality of different targets of interest with the same first nucleic acid-chromophore, wherein each of the first nucleic acid-chromophores labeling each of the plurality of different targets of interest is in a different nucleic acid assembly, wherein each of the different nucleic acid assemblies has a change relative to the other nucleic acid assemblies such that one or more of the photophysical properties of the adjacent chromophores is altered relative to those photophysical properties of the adjacent chromophores in the other nucleic acid assemblies, and detecting the photophysical properties of the adjacent chromophores in the different nucleic acid assemblies, thereby detecting the different targets of interest of the first plurality of different targets of interest.

The method can further include labeling each of a second plurality of different targets of interest with the same second nucleic acid-chromophore, wherein each of the second nucleic acid-chromophores labeling each of the second plurality of different targets of interest is in a different nucleic acid assembly, wherein each of the different nucleic acid assemblies has a change relative to the other nucleic acid assemblies such that one or more of the photophysical properties of the adjacent chromophores is altered relative to those photophysical properties of the adjacent chromophores in the other nucleic acid assemblies, and detecting the photophysical properties of the adjacent chromophores in the different nucleic acid assemblies, thereby detecting the different targets of interest of the second plurality of different targets of interest.

C. Alteration of Photophysical Properties of Nucleic Acid-Chromophore

The photophysical properties of a nucleic acid-chromophore can be changed by making a change in the nucleic acid assembly that contains the nucleic acid-chromophore.

The disclosed nucleic acid-chromophore and methods of using can be further understood through the following enumerated paragraphs.
1. A nucleic acid-chromophore including a nucleic acid strand including two or more adjacent chromophores, wherein, when the nucleic acid-chromophore is in a nucleic acid assembly, one or more photophysical properties of the adjacent chromophores is altered by a change in the nucleic acid assembly.
2. The nucleic acid-chromophore of paragraph 1, wherein the nucleic acid assembly includes a nucleic acid scaffold, wherein the change in the nucleic acid assembly is a change in the length of a nucleic acid hybrid in the nucleic acid scaffold that is opposite the adjacent chromophores.
3. The nucleic acid-chromophore of paragraph 1 or 2, wherein the nucleic acid assembly includes a DX tile, wherein the change in the nucleic acid assembly is a change in the length of a nucleic acid hybrid in the DX tile opposite the adjacent chromophores.
4. The nucleic acid-chromophore of any one of paragraphs 1-3, wherein one or more of the chromophores is a cyanine, a squaraine, a pentacene, or a perylene diimide.
5. The nucleic acid-chromophore of any one of paragraphs 1-4, wherein one or more of the chromophores is a cyanine, optionally wherein the cyanine is selected from an indocarbocyanine, an indodicarbocyanine and an indotricarbocyanine.
6. The nucleic acid-chromophore of any one of paragraphs 1-5, wherein the adjacent chromophores are cyanines, optionally wherein the adjacent cyanines are selected from indocarbocyanines, indodicarbocyanines and indotricarbocyanines.
7. The nucleic acid-chromophore of any one of paragraphs 1-6, wherein the nucleic acid strand is composed of deoxyribonucleotides (DNA), ribonucleotides (RNA), or analogs or modified nucleotides thereof.
8. The nucleic acid-chromophore of any one of paragraphs 1-7, wherein the nucleic acid strand is composed of deoxyribonucleotides (DNA).
9. The nucleic acid-chromophore of any one of paragraphs 1-7, wherein the nucleic acid strand is composed of locked nucleic acids (LNA) or peptide nucleic acids (PNA).
10. The nucleic acid-chromophore of any one of paragraphs 1-9, wherein the nucleic acid-chromophore is in a nucleic acid assembly, wherein the nucleic acid assembly is coupled to a biological or nonbiological material.
11. The nucleic acid-chromophore of paragraph 10, wherein the nucleic acid assembly is operably coupled to the biological or nonbiological material, wherein interaction of a molecule of interest with the biological or nonbiological material produces the change in the nucleic acid assembly.
12. The nucleic acid-chromophore of any one of paragraphs 1-11, wherein the one or more photophysical properties of the adjacent chromophores includes the quantum yield of the adjacent chromophores.
13. The nucleic acid-chromophore of any one of paragraphs 1-12, wherein the one or more photophysical properties of the adjacent chromophores includes the energy dependent optical density of the adjacent chromophores.
14. The nucleic acid-chromophore of any one of paragraphs 1-13, wherein the one or more photophysical properties of the adjacent chromophores includes the emission energetic profile of the adjacent chromophores.
15. The nucleic acid-chromophore of any one of paragraphs 1-14, wherein the one or more photophysical properties of the adjacent chromophores includes the excited state lifetime of the adjacent chromophores.
16. The nucleic acid-chromophore of any one of paragraphs 1-15, wherein the one or more photophysical properties of the adjacent chromophores includes the excited state lifetime of the adjacent chromophores, wherein the excited state lifetime of the adjacent chromophores is altered by a change in solvent polarity.
17. The nucleic acid-chromophore of any one of paragraphs 1-16, wherein the alteration in the one or more photophysical properties of the adjacent chromophores is sufficient to distinguish a single altered nucleic acid assembly from a single unaltered nucleic acid assembly.
18. The nucleic acid-chromophore of any one of paragraphs 1-17, wherein the nucleic acid assembly is encapsulated.
19. The nucleic acid-chromophore of any one of paragraphs 1-18, wherein the nucleic acid assembly is encapsulated in an organic or inorganic material.
20. The nucleic acid-chromophore of any one of paragraphs 1-19, wherein the nucleic acid assembly is encapsulated in an organic material.
21. The nucleic acid-chromophore of any one of paragraphs 1-19, wherein the nucleic acid assembly is encapsulated in an inorganic material.
22. The nucleic acid-chromophore of paragraph 21, wherein the inorganic material is silica.
23. A method of detecting a change in a nucleic acid assembly, wherein the nucleic acid assembly includes the nucleic acid-chromophore of any one of paragraphs 1-22, wherein the method includes measuring one or more of the photophysical properties of the adjacent chromophores, wherein the change is detected if one or more of the measured photophysical properties of the adjacent chromophores is altered compared to a reference photophysical property.
24. The method of paragraph 23, wherein the reference photophysical property is the photophysical property of the nucleic acid assembly in the absence of a change in the nucleic acid assembly.
25. The method of paragraph 23 or 24, wherein the change in the nucleic acid assembly is produced by interaction of a molecule of interest with a biological or nonbiological material to which the nucleic acid assembly is operably coupled.
26. The method of paragraph 23 or 24, wherein the change in the nucleic acid assembly is produced by correct assembly of a scaffold origami of which the nucleic acid assembly becomes a part.
27. The method of paragraph 23 or 24, wherein the change in the nucleic acid assembly is produced by a change in the milieu of the nucleic acid assembly.
28. The method of paragraph 27, wherein the change in milieu of the nucleic acid assembly is a change in the polarity of solvent in which the nucleic acid assembly is dissolved or suspended.
29. A method of multiplex detection, the method including:
labeling each of a first plurality of different targets of interest with the same first nucleic acid-chromophore of any one of paragraphs 1-22, wherein each of the first nucleic acid-chromophores labeling each of the plurality of different targets of interest is in a different nucleic acid assembly, wherein each of the different nucleic acid assemblies has a change relative to the other nucleic acid assemblies such that one or more of the photophysical properties of the adjacent chromophores is altered relative to those photophysical properties of the adjacent chromophores in the other nucleic acid assemblies, and detecting the photophysical properties of the adjacent chromophores in the different nucleic acid assemblies, thereby detecting the different targets of interest of the first plurality of different targets of interest.
30. The method of paragraph 29 further including:
labeling each of a second plurality of different targets of interest with the same second nucleic acid-chromophore of any one of paragraphs 1-22, wherein each of the second nucleic acid-chromophores labeling each of the second plurality of different targets of interest is in a different nucleic acid assembly, wherein each of the different nucleic acid assemblies has a change relative to the other nucleic acid assemblies such that one or more of the photophysical properties of the adjacent chromophores is altered relative to those photophysical properties of the adjacent chromophores in the other nucleic acid assemblies, and detecting the photophysical properties of the adjacent chromophores in the different nucleic acid assemblies, thereby detecting the different targets of interest of the second plurality of different targets of interest.
31. A method of altering one or more photophysical properties of a nucleic acid-chromophore, the method including
making a change in a nucleic acid assembly, wherein the nucleic acid assembly includes the nucleic acid chromophore, wherein the nucleic acid chromophore includes a nucleic acid strand including two or more adjacent chromophores, wherein the one or more photophysical properties of the nucleic acid-chromophore are altered by the change in the nucleic acid assembly.
32. The method of paragraph 31, wherein the nucleic acid assembly includes a nucleic acid scaffold, wherein the change in the nucleic acid assembly is a change in the length of a nucleic acid hybrid in the nucleic acid scaffold that is opposite the adjacent chromophores.
33. The method of paragraph 31 or 32, wherein the nucleic acid assembly includes a DX tile, wherein the change in the nucleic acid assembly is a change in the length of a nucleic acid hybrid in the DX tile opposite the adjacent chromophores.
34. The method of any one of paragraphs 31-33, wherein one or more of the chromophores is a cyanine, a squaraine, a pentacene, or a perylene diimide.
35. The method of any one of paragraphs 31-34, wherein one or more of the chromophores is a cyanine, optionally wherein the cyanine is selected from an indocarbocyanine, an indodicarbocyanine and an indotricarbocyanine.
36. The method of any one of paragraphs 31-35, wherein the adjacent chromophores are cyanines, optionally wherein the adjacent cyanines are selected from indocarbocyanines, indodicarbocyanines and indotricarbocyanines.
37. The method of any one of paragraphs 31-36, wherein the nucleic acid strand is composed of deoxyribonucleotides (DNA), ribonucleotides (RNA), or analogs or modified nucleotides thereof.
38. The method of any one of paragraphs 31-37, wherein the nucleic acid strand is composed of deoxyribonucleotides (DNA).
39. The method of any one of paragraphs 31-37, wherein the nucleic acid strand is composed of locked nucleic acids (LNA) or peptide nucleic acids (PNA).
40. The method of any one of paragraphs 31-39, wherein the nucleic acid-chromophore is in a nucleic acid assembly, wherein the nucleic acid assembly is coupled to a biological or nonbiological material.
41. The method of paragraph 40, wherein the nucleic acid assembly is operably coupled to the biological or nonbiological material, wherein interaction of a molecule of interest with the biological or nonbiological material produces the change in the nucleic acid assembly.
42. The method of any one of paragraphs 31-41, wherein the one or more photophysical properties of the adjacent chromophores includes the quantum yield of the adjacent chromophores.
43. The method of any one of paragraphs 31-42, wherein the one or more photophysical properties of the adjacent chromophores includes the energy dependent optical density of the adjacent chromophores.
44. The method of any one of paragraphs 31-43, wherein the one or more photophysical properties of the adjacent chromophores includes the emission energetic profile of the adjacent chromophores.
45. The method of any one of paragraphs 31-44, wherein the one or more photophysical properties of the adjacent chromophores includes the excited state lifetime of the adjacent chromophores.
46. The method of any one of paragraphs 31-45, wherein the one or more photophysical properties of the adjacent chromophores includes the excited state lifetime of the adjacent chromophores, wherein the excited state lifetime of the adjacent chromophores is altered by a change in solvent polarity.
47. The method of any one of paragraphs 31-46, wherein the alteration in the one or more photophysical properties of the adjacent chromophores is sufficient to distinguish a single altered nucleic acid assembly from a single unaltered nucleic acid assembly.
48. The method of any one of paragraphs 31-47, wherein the nucleic acid assembly is encapsulated.
49. The method of any one of paragraphs 31-48, wherein the nucleic acid assembly is encapsulated in an organic or inorganic material.
50. The method of any one of paragraphs 31-49, wherein the nucleic acid assembly is encapsulated in an organic material.
51. The method of any one of paragraphs 31-49, wherein the nucleic acid assembly is encapsulated in an inorganic material.
52. The method of paragraph 51, wherein the inorganic material is silica.

### EXAMPLES

### Example 1. Alternation of Photophysical Properties in DNA-Chromophore Assemblies by Changing Assembles Structure and/or Local Environment

Materials and Methods

Construction and Characterization of Cy3 Modified DNA Conjugate

Nucleoside and Cyanine 3 (Cy3) phosphoramidite for oligonucleotide synthesis were purchased from Glen Research. DNA solid phase synthesis was carried on a Dr. Oligo synthesizer following standard oligonucleotide solid phase synthesis procedures. An extended coupling time (3 min) was used for Cy3 phosphoramidite. The Cy3 modified DNA strands were purified by HPLC. Unmodified oligonucleotides were purchased from Integrated DNA Technologies. Constructs were formed by mixing a stoichiometric quantity of each strand, as estimated by OD260. The samples in 1XTAE Mg (40 mM Tris-HCl, 20 mM acetic acid, 2 mM EDTA and 12.5 mM magnesium acetate) buffer were sealed in PCR tubes and then incubated by the following annealing protocol: from 65. C to 20. C in 2 h. Purification was verified through non-denaturing gel electrophoresis. The chemical structure of Cy3 dimer and the schematics showing the Xy3-DX tiles are shown in FIGS. 1A-1D and FIG. 2A-2D. Linear absorbance spectra were acquired using Shimadzu UV-2401PC spectrometer. Excitation and emission spectra were acquired using a Varian Cary Elipse. Quantum yield was calculated relative to rhodamine 6G.

Spectroscopic Characterization

Ensemble time resolved emission spectra were collected using a pulsed excitation scheme. Pulsed were generated by passing the 800 nm output of a Ti:Sapphire oscillator through a photonic crystal fiber (NKT photonics FemtoWhite 800). Excitation wavelengths were selected with bandpass filters. Photons were detected on a single photon avalanche diode and temporally tagged using a time-correlation module (Picoquant, PicoHarp 300). Further details are provided below.

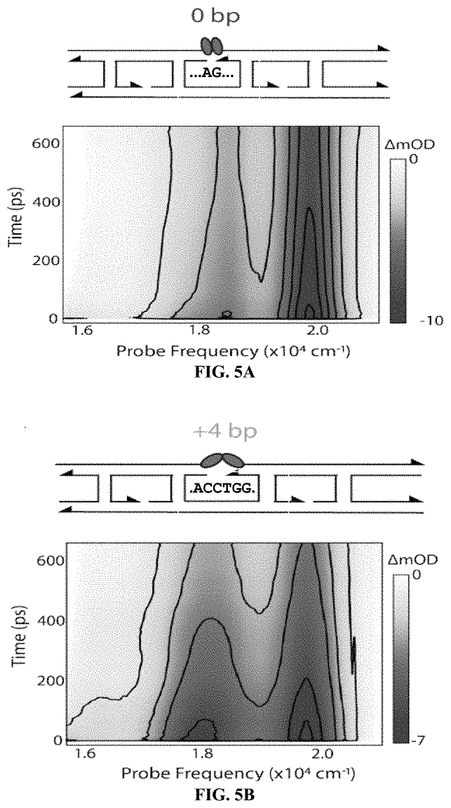

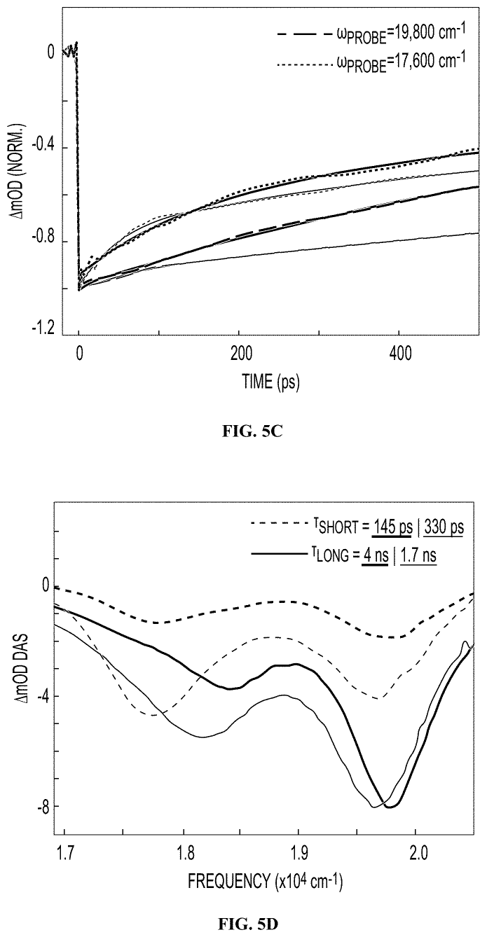

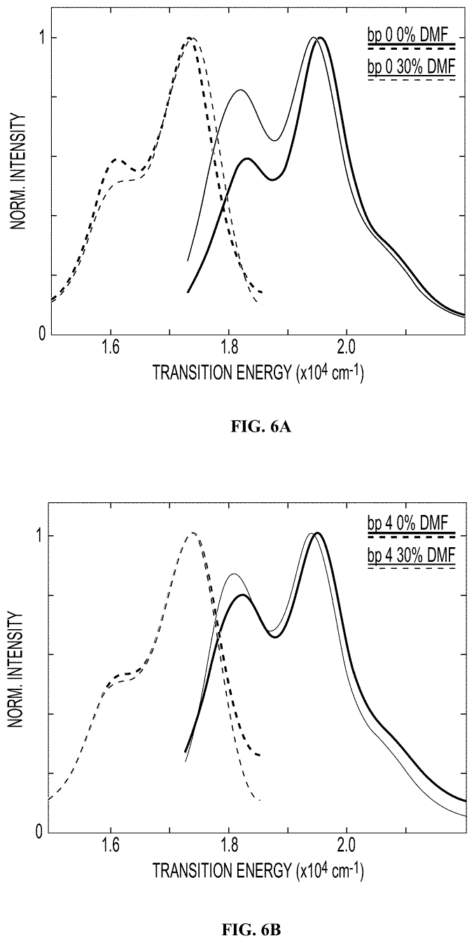

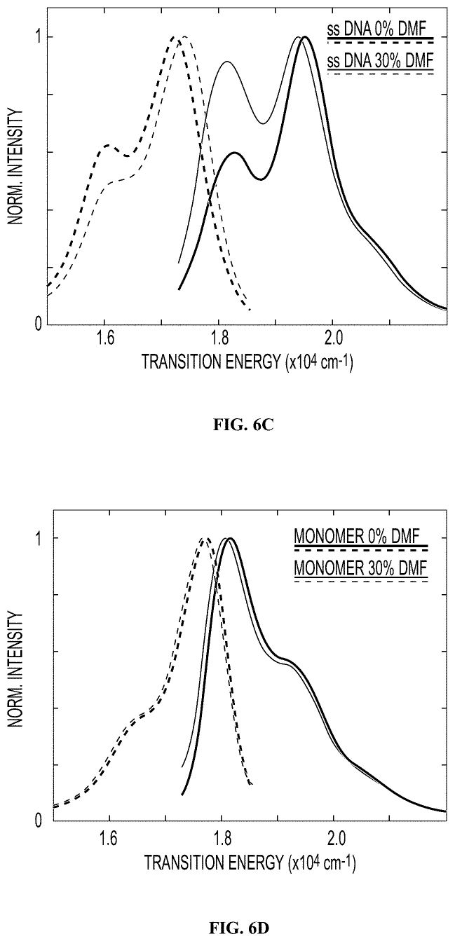

Femtosecond transient broadband absorption studies were conducted to collect the data shown in FIG. 5A-5D. The set-up is described in detail elsewhere. Briefly, the output of a Ti:sapphire regenerative amplifier (Coherent Libra, 5 kHz) was used to generate a white light supercontinuum by passing the 800 nm light through a pressurized argon gas chamber (20 psi). Pulses were compressed to a duration of 12 fs using two sets of chipped mirrors (Ultrafast Innovations GmbH, −40 fs2/mm GVD compensation per double bounce) and spectrally filtered to a center wavelength of 540 nm (6 nJ/pulse). Spectra were collected on a line CCD (e2v AViiVA) coupled to a 2.5 kHz chopper to collect spectra with the “pump on” and “pump off.”

Single-molecule fluorescence spectroscopy was performed on surface immobilized DNA samples. Samples were prepared on a quartz coverslip via aminosilane functionalization and a 2% biotin-PEGylated surface. DNA samples were measured in an aqueous buffer comprised of 1×TAE and the triplet and oxygen scavengers (2 mM Trolox, Sigma-Aldrich, and 2.5 mM protocatechuic acid and 25 nM protocatechuic-3,4-dioxygenase). Experiments were performed using a home-build confocal microscope described in detail elsewhere. Briefly, excitation light was generated using an 800 nm pumped (Coherent Vitara-S) photonic crystal fiber (NKT Photonics, FemtoWhite) and filtered to 550 nm (Omega, RPB 540/560, T>90% for 545-555 nm). An oil-immersion objective (Olympus; UPLSAPO100XO, NA=1.4) was used to focus the excitation to the sample and collect the resultant fluorescence. Emitted photons were selected a dichroic filter (Laser 2000, SP01-561RU) with an 590 nm bandpass filter (Semrock, 590/36-25) and detected on an avalanche photodiode (Excelitas, SPCM-AQRH-15) in conjunction with a single photon counting device (Picoquant, PicoHarp 300).

Non-Denaturing Gel Electrophoresis Analysis

1 μL of 10×gel loading buffer was added to 9 μL of annealed DNA constructs and loaded to 10% polyacrylamide gels. The gels were run at room temperature in 60 V. The gels were stained with stains-all solution or directly imaged with blue-light imager afterward. The images of non-denaturing gel electrophoresis of 0 to +5 bp Cy3-DX samples indicate the successful formation of 0 to +4 bp constructs, whereas +5 bp construct show up as multiple species, and successful formation of 0 to −5 bp constructs.

DX Tile Design

Strand construction of all DX tile scaffolds for the Cy3 dimers is shown in FIGS. 2A-2D. The associated strand sequences are reported in Tables 1 and 2.

TABLE 1
DNA sequences for DX tile
construction shown in FIG. 2A.

| | | SEQ |
|---|---|---|
| Strand | Sequence | ID NO: |
| A | 5′-CGCCATCCTAGCCGCTATGTCATTGA- | 1 |
| | Cy3-Cy3-CGAGCTGTCGAGCATGTGAGCA | |
| | TTGA-3′ | |
| B | 5′-GGCAATCAGATCGACGTGACGCTACAG | 2 |
| | AGCAGTCTATCAGGCGAGTTGGTCG-3′ | |
| C | 5′-TCAATTGCTCTGTAGGCTCG-3′ | 3 |
| 1 | 5′-CGACCAACTCTAGGATGACG-3′ | 4 |
| 2 | 5′-TAGACGACATAGCGGCGCCTGA-3′ | 5 |
| 3 | 5′-GTCGACATGCTCGACACGTCAC-3′ | 6 |
| 4 | 5′-TCAATGCTCATCTGATTGCC-3′ | 7 |

TABLE 2
DNA sequences for DX tile construction for the
addition and removal of complementary base pairs
(structures shown in FIGS. 2B and 2C).

| | | | SEQ ID |
|---|---|---|---|
| Construct | Strand | Sequence | NO: |
| +1 bp | B* | 5′-GGCAATCAGATCGACG | 8 |
| | | TGACGCTACAGGAGCAGTC | |
| | | TATCAGGCGAGTTGGTC | |
| | | G-3′ | |
| +1 bp | C* | 5′-TCAATTGCTCCTGTAG | 9 |
| | | GCTCG-3′ | |
| +2 bp | B* | 5′-GGCAATCAGATCGACG | 10 |
| | | TGACGCTACATGGAGCAGT | |
| | | CTATCAGGCGAGTTGGTC | |
| | | G-3′ | |
| +2 bp | C* | 5′-TCAATTGCTCCATGTA | 11 |
| | | GGCTCG-3′ | |
| +3 bp | B* | 5′-GGCAATCAGATCGACG | 12 |
| | | TGACGCTACACTGGAGCAG | |
| | | TCTATCAGGCGAGTTGGTC | |
| | | G-3′ | |
| +3 bp | C* | 5′-TCAATTGCTCCAGTGT | 13 |
| | | AGGCTCG-3′ | |
| +4 bp | B* | 5′-GGCAATCAGATCGACG | 14 |
| | | TGACGCTACACCTGGAGCA | |
| | | GTCTATCAGGCGAGTTGGT | |
| | | CG-3′ | |
| +4 bp | C* | 5′-TCAATTGCTCCAGGTG | 15 |
| | | TAGGCTCG-3′ | |
| +5 bp | B* | 5′-GGCAATCAGATCGACG | 16 |
| | | TGACGCTACAACCTGGAGC | |
| | | AGTCTATCAGGCGAGTTGG | |
| | | TCG-3′ | |
| +5 bp | C* | 5′-TCAATTGCTCCAGGTT | 17 |
| | | GTAGGCTCG-3′ | |
| −2 bp | B* | 5′-GGCAATCAGATCGACG | 18 |
| | | TGACGCTACAGCAGTCTAT | |
| | | CAGGCGAGTTGGTCG-3′ | |
| −2 bp | C* | 5′-TCAATTGCTGTAGGCT | 19 |
| | | CG-3′ | |
| −3 bp | B* | 5′-GGCAATCAGATCGACG | 20 |
| | | TGACGCTAAGCAGTCTATC | |
| | | AGGCGAGTTGGTCG-3′ | |
| −3 bp | C* | 5′-TCAATTGCTTAGGCTC | 21 |
| | | G-3′ | |
| −4 bp | B* | 5′-GGCAATCAGATCGACG | 22 |
| | | TGACGCTAGCAGTCTATCA | |
| | | GGCGAGTTGGTCG-3′ | |
| −4 bp | C* | 5′-TCAATTGCTAGGCTC | 23 |
| | | G-3′ | |
| −5 bp | B* | 5′-GGCAATCAGATCGACG | 24 |
| | | TGACGCTGCAGTCTATCAG | |
| | | GCGAGTTGGTCG-3′ | |
| −5 bp | C* | 5′-TCAATTGCAGGCTC | 25 |
| | | G-3′ | |

Time Resolved Emission Spectroscopy

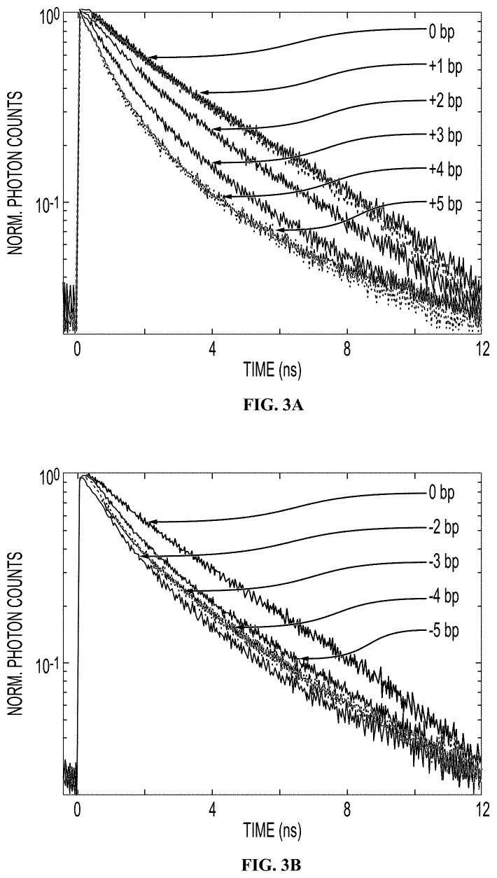

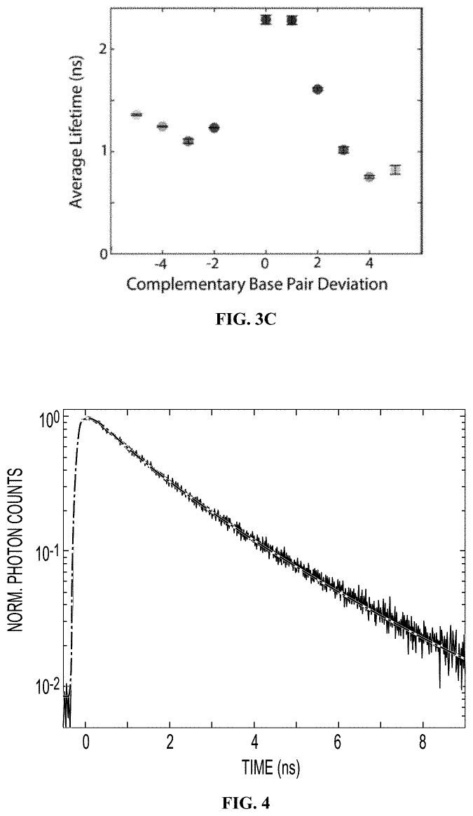

Ensemble time resolved emission data shown in FIGS. 3A-3C were collected using a pulsed 520 nm excitation scheme with single photon detection. Pump pulses were generated using a Ti:Sapphire laser source (Coherent Vitara-S) passed through a photonic crystal fiber (NKT photonics FemtoWhite 800) to generate a broadband continuum. The excitation wavelength was selected using a 520 nm bandpass filter (Thorlabs 1-B520-10). The laser was attenuated to 15 pJ/pulse using neutral density filters. Emitted photons were selected using a 580 nm bandpass filter (Thorlabs FB580-10). Single photons were detected on a single photon avalanche diode (Micro Photon Devices) in conjunction with a time-correlation module (Picoquant, PicoHarp 300). Histogrammed traces were fit to a two component exponential decay convolved with an experimentally-measured 60 ps instrument response function.

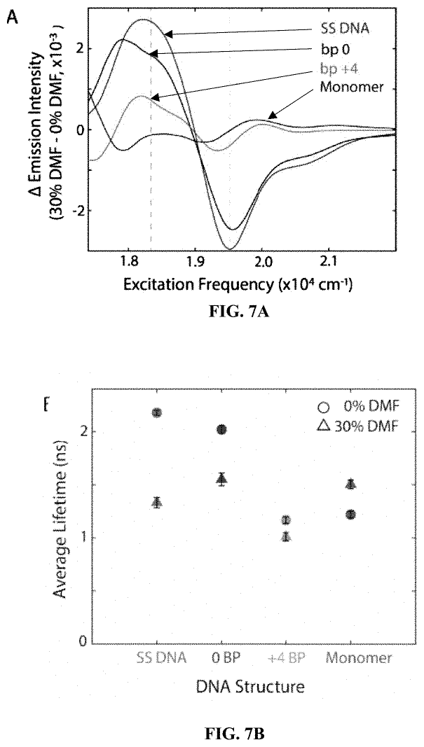

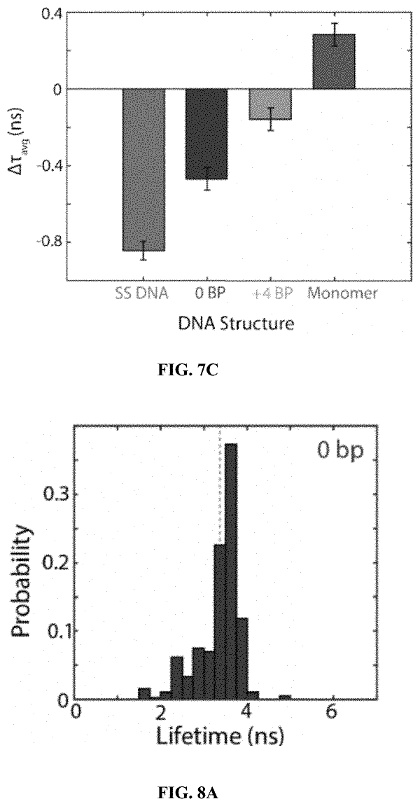

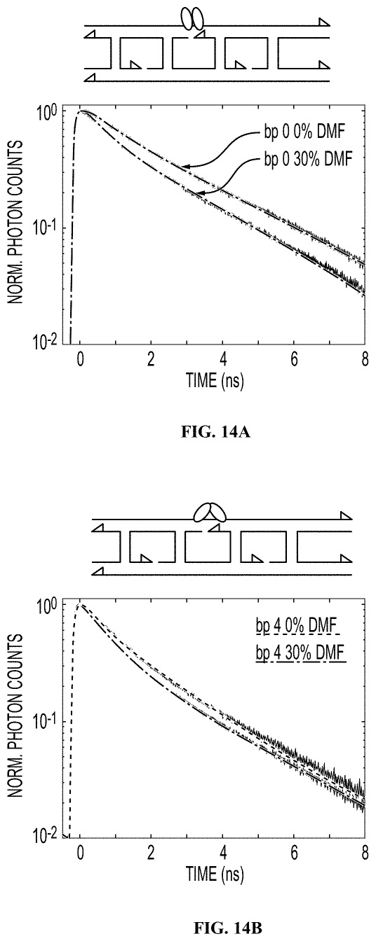

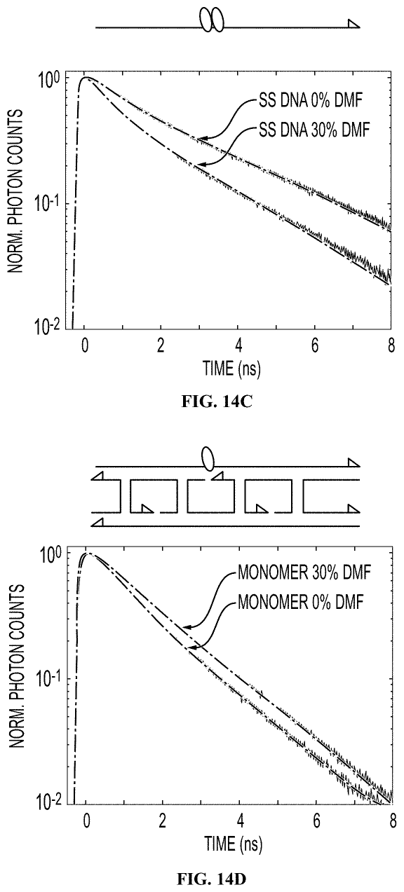

Ensemble time resolved emission data examining solvent dependent behavior shown in main text FIGS. 7A-7C and FIGS. 14A-14D were collected using a pulsed 550 nm excitation scheme with single photon detection. The data shown in FIGS. 14A-14D is summarized in Table 6.

TABLE 6
Solvent dependent TCSPC traces (curves
shown in FIGS. 14A-14D).

| | A₁ | τ₁ | A₂ | τ₂ |
|---|---|---|---|---|
| 0 bp 0% DMF | 0.61 | 2.93 ns | 0.39 | 0.57 ns |
| 0 bp 30% DMF | 0.52 | 2.62 ns | 0.48 | 0.54 ns |
| +4 bp 0% DMF | 0.48 | 1.93 ns | 0.52 | 0.46 ns |
| +4 bp 30% DMF | 0.39 | 1.89 ns | 0.61 | 0.44 ns |
| SS DNA 0% DMF | 0.57 | 3.38 ns | 0.43 | 0.58 ns |
| SS DNA 30% DMF | 0.43 | 2.46 ns | 0.57 | 0.46 ns |
| Monomer 0% DMF | 0.48 | 1.77 ns | 0.52 | 0.73 ns |
| Monomer 30% DMF | 0.56 | 1.97 ns | 0.44 | 0.91 ns |

Broadband continuum pulses were generated by passing an 800 nm Ti-Sapphire pulse (Spectra Physics Mai Tai) through a photonic crystal fiber (NKT Pho-tonics FemtoWhite 800). The excitation wavelength was selected using a 550 nm bandpass filter (Thorlabs ET550/15ex) and emitted photons were selected using a 580 nm bandpass filter (Thorlabs FB580-10). The laser was attenuated to 12 pJ/pulse using neutral density filters. Photons were detected on a single photon avalanche diode (Micro Photon Devices) in conjunction with a time-correlation module (Picoquant, PicoHarp 300). The photon arrival times were used to construct histograms for each measurement. Fluorescence life times were extracted by fitting the histograms to a biexpoential decay convolved with the experimentally-measured 80 ps FWHM instrument response function (IRF):

$$I ( t ) = {A}_{0} + IRF ⊗ {\sum }_{i = 1}^{2} {A}_{i} exp ( - t / {𝒯}_{i} ) \qquad ( 1 )$$

Broadband Transient Absorption Spectroscopy

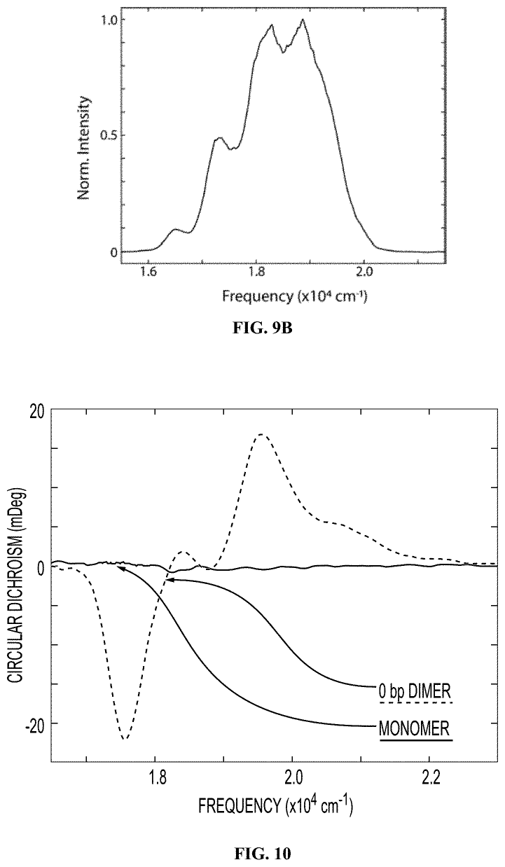

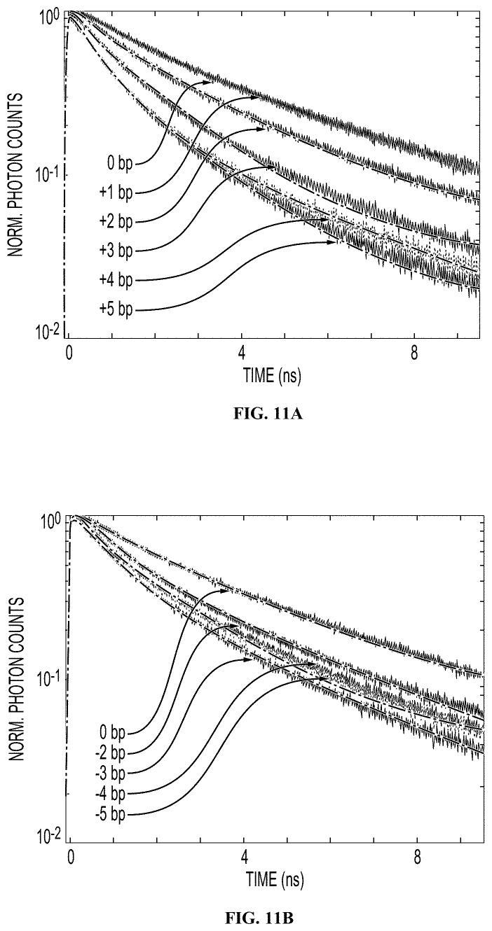

The cross-correlation for the pump and probe pulses and the pump spectral profile from the broadband transient absorption experiments are shown in FIG. 9. The autocorrelation of the pump pulse showed a FWHM of 12 fs. The cross correlation between the pump and probe showed a FWHM of 190 fs. The probe dispersion was corrected for in the global analysis fitting.

Single-Molecule Fluorescence Spectroscopy

For single molecule fluorescence experiments, histograms of the delay time for the detected lifetime were constructed to recover the associated emissive lifetimes. The kinetic traces were fit using maximum likelihood estimation (MLE) reconvolution (with the 390 ps IRF) to recover the monoexponential lifetime components, as shown below.

$$F ( t ) = {N}_{tot} [ ( 1 - {γ}_{F} ) \frac{{A}_{0} + IRF ⊗ [ {A}_{i} exp ( - t / {𝒯}_{i} ) ]}{{\int }_{0 ns}^{12.5 ns} \{ {A}_{0} + IRF ⊗ [ {A}_{i} exp ( - t / {𝒯}_{i} ) ] \} dt} + {γ}_{F} \frac{BG ( t )}{{\int }_{0 ns}^{12.5 ns} BG ( t ) dt} ] \qquad ( 2 )$$

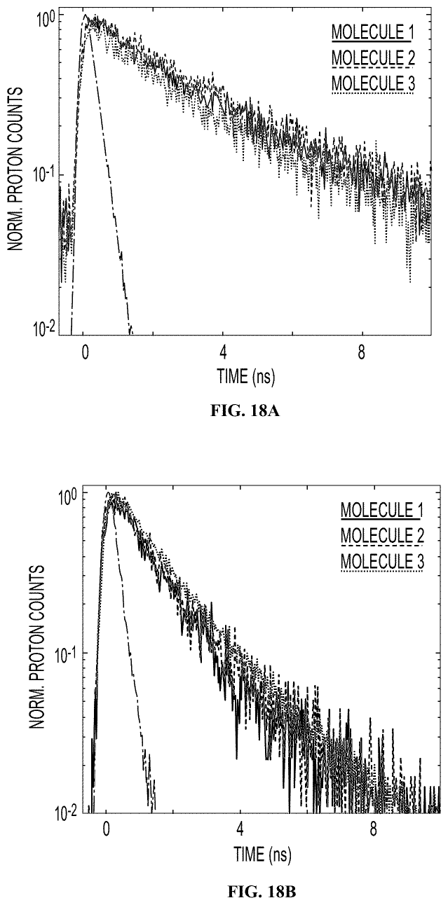

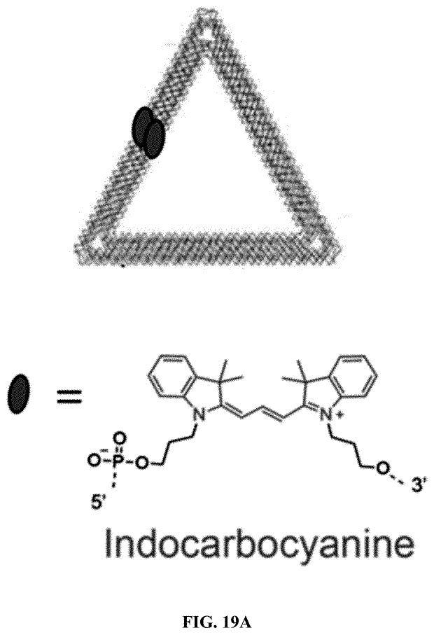

The fraction of background signal is represented by γF. Background signal, BG(t), was collected in a region of the sample substrate with no molecules. The background-subtracted photon count of the trace is given by Ntot. The decay lifetime is given by T. Representative single molecule fluorescence traces are shown in FIGS. 18A-18B. Data were fit to 5000 photon segments. Each histogram corresponds to 75-130 individual molecules.

For the phasor analysis, time domain collected emission lifetimes recovered from the histogrammed photon arrival times are formally transformed to the phasor plot by the following two relations.

$$G ( t ) = \frac{{\int }_{0}^{∞} I ( t ) cos ( ω t ) dt}{{\int }_{0}^{∞} I ( t ) dt} \qquad ( 3 )$$

$$S ( t ) = \frac{{\int }_{0}^{∞} I ( t ) sin ( ω t ) dt}{{\int }_{0}^{∞} I ( t ) dt} \qquad ( 4 )$$

Here I(t) is the decay profile and to refers to the laser repetition frequency. Due to the the finite duration of the instrument response function, the theoretical decay profile, I(t) theo(t), can be related to the experimentally recovered decay profile, I(t)exp(t), through convolution with the instrument response function.

I(t)ₑₓₚ=I(t)ₜₕₑₒ⊗IRF(t) (5)

This can be solved by using a reference compound to rescale the phasor vector.

$$[ \begin{matrix}{G}_{theo} \\ {S}_{theo}\end{matrix} ] = \frac{1}{{G}_{ref}^{2} + {S}_{ref}^{2}} [ \begin{matrix}{G}_{ref} & {S}_{ref} \\ - {S}_{ref} & {G}_{ref}\end{matrix} ] [ \begin{matrix}{G}_{exp} \\ {S}_{exp}\end{matrix} ] \qquad ( 6 )$$

Here the instrument response function was used as the reference compound, however similar results were achieved using rhodamine 6G as a reference compound at the ensemble level.

### Results and Discussion

Construction and Characterization of Cy3 Dimers

Dimers of indocarbocyanine (Cy3) were incorporated into DNA strands using phosphoramidite chemistry (FIG. 1A). DX tiles were assembled by annealing dimer-containing scaffold strands with staple strands. Separation of the folded DX tile construct was confirmed through PAGE gel separation. The standard DX tile (FIG. 1B) contains a central crossover strand with 20 base pairs (denoted as “0 bp”). The DX tiles designed to pull the Cy3 dimers apart (FIG. 1C) contain a central crossover strand with 21-25 bases (“+1 to +5 bp”), while DX tiles designed to push the Cy3 dimers together (FIG. 1D) contain a central crossover strand with 15-18 bases (“−5 to −2 bp”).

The geometries of the Cy3 dimers were initially investigated by comparing their linear absorption spectra. The spectra for all the dimers are shown in FIGS. 1E-1F. The spectra exhibited a redistribution of oscillator strength from the 0-0 to the 0-1 band as well as a hypsochromatic shift in the transition energy for the 0 bp Cy3 dimer in comparison to the monomer (dark gray). Both of these features are spectroscopic signatures of a vibronically coupled subradiant “H-type” aggregate, which is generated through cofacial stacking of the chromophores. The quantum yields (Table 4) were also lower than the monomer, consistent with the subradiant nature of the H-type dimer. In FIGS. 1G-1H, the circular dichroism spectra of the 0 bp Cy3 dimers show a bisignate line shape, where the signs of the observed peaks have been assigned to a vibronically coupled H-aggregate. The formation of an H-type dimer is consistent with previous findings that Cy3 dimers on the same strand of DNA stack largely cofacially.

The geometric changes that arise from the addition or removal of base pairs from the central crossover strand (thus tuning the adjacent-DX spacing) appear spectroscopically as a spectral shift of, and decrease in the relative intensity of, the 0-1 band (FIGS. 1E-1F, 1I-1K, Tables 3 and 4), indicating a decrease in electronic coupling. Consistent with this assignment, the intensity of the circular dichroism spectra decreases upon addition or removal of base pairs (FIGS. 1G-1H). The disruption of the structure for the dimer chiral response points to a change in stacking contributing to the measured spectroscopic signatures, particularly for the “pull”-type dimer where changes in the spectral profiles are observed for the +3 bp to +5 bp constructs. The observed spectral changes can arise from a combination of a decrease in electronic coupling and a transition from cofacial stacking to head-to-tail stacking, which is known as a “J-type” dimer. Despite the spectral signatures of a J-type dimer, several of the dimers (+3 bp, +4 bp, +5 bp, −2 bp, −3 bp) showed a decreased quantum yield, which can be a result of aggregation-induced nonradiative decay pathways.

TABLE 3
Absorption profile ν00 and ν01 energies (spectra given in FIGS.
1E-1H) and band intensity ratios (I00/01) for DX tile constructs.

| | ν₀₀ | ν₀₁ | I_{00/01} | ϵₘₐₓ |
|---|---|---|---|---|
| 0 bp | 18,350 cm⁻¹ | 19,570 cm⁻¹ | 0.53 | 140,400 M⁻¹cm⁻¹ |
| +1 bp | 18,350 cm⁻¹ | 19,570 cm⁻¹ | 0.55 | 135,400 M⁻¹cm⁻¹ |
| +2 bp | 18,350 cm⁻¹ | 19,570 cm⁻¹ | 0.58 | 147,700 M⁻¹cm⁻¹ |
| +3 bp | 18,350 cm⁻¹ | 19,610 cm⁻¹ | 0.61 | 132,700 M⁻¹cm⁻¹ |
| +4 bp | 18,320 cm⁻¹ | 19,610 cm⁻¹ | 0.65 | 132,800 M⁻¹cm⁻¹ |
| +5 bp | 18,280 cm⁻¹ | 19,570 cm⁻¹ | 0.72 | 125,900 M⁻¹cm⁻¹ |
| −2 bp | 18,320 cm⁻¹ | 19,570 cm⁻¹ | 0.64 | 131,900 M⁻¹cm⁻¹ |
| −3 bp | 18,280 cm⁻¹ | 19,570 cm⁻¹ | 0.72 | 125,010 M⁻¹cm⁻¹ |
| −4 bp | 18,210 cm⁻¹ | 19,530 cm⁻¹ | 0.77 | 119,400 M⁻¹cm⁻¹ |
| −5 bp | 18,210 cm⁻¹ | 19,490 cm⁻¹ | 0.78 | 113,700 M⁻¹cm⁻¹ |
| Mon. | 18,180 cm⁻¹ | 19,380 cm⁻¹ | 1.76 | 129,800 M⁻¹cm⁻¹ |

TABLE 4
Emission profile ν00 and ν01 energies (spectra given in FIGS.
1E-1H) and band intensity ratios (I00/01) for DX tile constructs.

| | ν₀₀ | ν₀₁ | I_{00/01} | Quantum Yield |
|---|---|---|---|---|
| 0 bp | 16,080 cm⁻¹ | 17,330 cm⁻¹ | 0.71 | 0.168 ± 0.008 |
| +1 bp | 16,080 cm⁻¹ | 17,330 cm⁻¹ | 0.73 | 0.165 ± 0.007 |
| +2 bp | 16,080 cm⁻¹ | 17,330 cm⁻¹ | 0.70 | 0.159 ± 0.002 |
| +3 bp | 16,100 cm⁻¹ | 17,330 cm⁻¹ | 0.68 | 0.101 ± 0.003 |
| +4 bp | 16,160 cm⁻¹ | 17,360 cm⁻¹ | 0.66 | 0.070 ± 0.001 |
| +5 bp | 16,160 cm⁻¹ | 17,360 cm⁻¹ | 0.64 | 0.113 ± 0.005 |
| −2 bp | 16,100 cm⁻¹ | 17,360 cm⁻¹ | 0.65 | 0.111 ± 0.007 |
| −3 bp | 16,180 cm⁻¹ | 17,390 cm⁻¹ | 0.6 | 0.125 ± 0.003 |
| −4 bp | 16,130 cm⁻¹ | 17,360 cm⁻¹ | 0.61 | 0.174 ± 0.005 |
| −5 bp | 16,160 cm⁻¹ | 17,360 cm⁻¹ | 0.61 | 0.168 ± 0.007 |
| Mon. | 16,640 cm⁻¹ | 17,780 cm⁻¹ | 0.36 | 0.274 ± 0.006 |

Dimer Geometry Tunes Photophysics

Structural distortions, such as those induced by the addition and removal of base pairs to the central crossover strand, vary the dynamics of the electronic excited states, which are responsible for many of the applications of the DNA-chromophore assembles. To characterize the overall excited state temporal evolution, time correlated single photon counting (TCSPC) was used to measure the emissive lifetime as shown in FIGS. 3A-3C. The 0 bp dimer exhibits a biexponential decay with a 0.28±0.02 ns and a 3.40±0.05 ns component, which gave an average lifetime of 2.2 ns. The two components likely correspond to static and/or dynamic sub-populations within the DNA scaffold with different geometries and thus different electronic couplings. Work on both Cy3 and Cy5 dimers formed on opposite strands of DNA has identified sub-populations of both J and H-type dimers, while Cy3 dimers formed on the same strand as in this work appear to have strongly coupled H-type populations, and weak H-type or weak J-type populations.

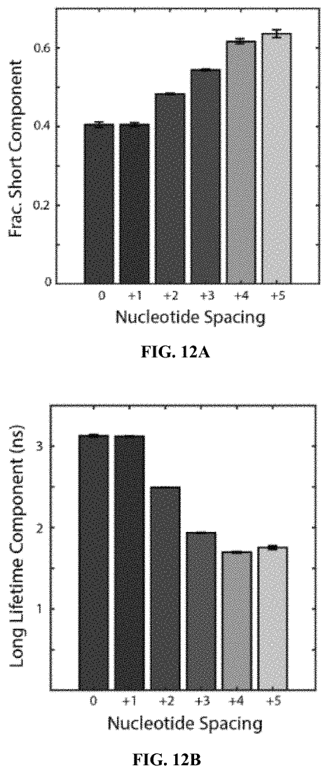

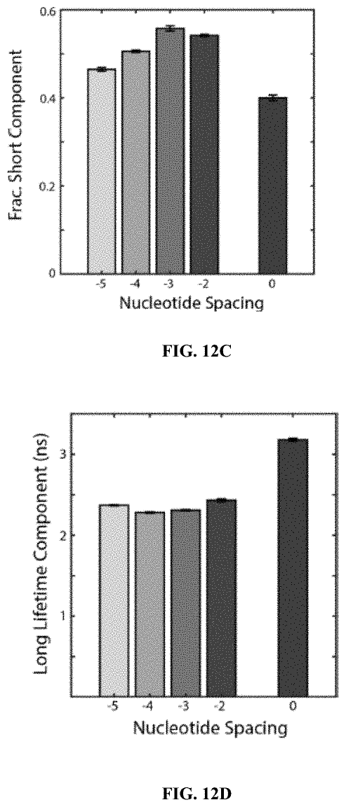

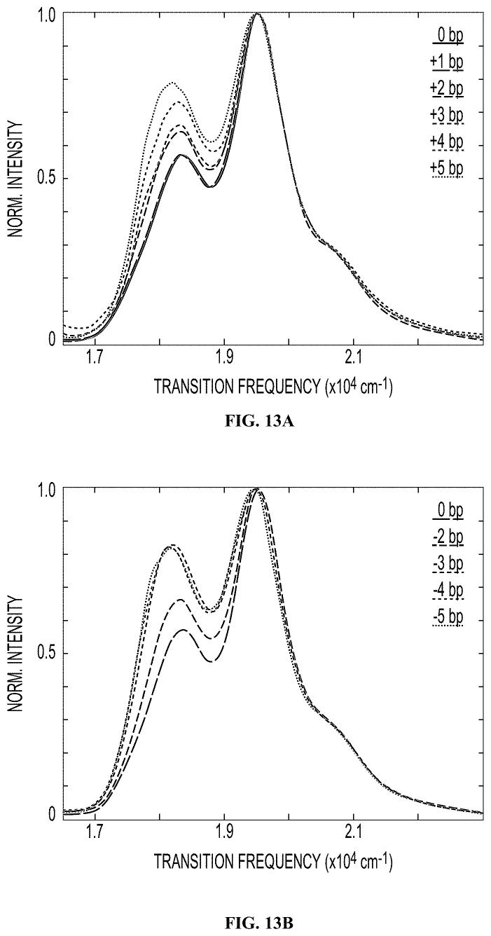

The Cy3 monomer has an average lifetime of 1.5 ns (FIG. 4). A transition from cofacial to head-to-tail stacking decreases the fluorescence lifetime due to the subradiant and superradiant nature of the H- and J-dimers, respectively. The addition or removal of complementary staple strand bases lowered the average lifetime from 2.2 ns to a minimum of 0.8 ns as shown in FIGS. 3A-3C. The lower values arise from the long-lifetime component shortening from 3.4 to 1.7 ns and the short-lifetime component increasing in amplitude (FIGS. 12A-12D and Table 5). The longer average lifetimes are above the monomer value whereas the shorter ones are below, strongly implying electronic coupling is retained with geometric changes across the dimer series. While the steady state spectra (FIGS. 1D-1G) indicated the electronic coupling decreases with the addition or removal of base pairs, these changes in lifetime along with the redistribution of oscillator strength indicate that the decrease in the magnitude of electronic coupling is accompanied by a geometric change into dimers with some J-type character.

TABLE 5
Parameters for TCSPC trace fitting for FIGS. 3A-
3C. Traces and fits shown in FIGS. 11A-11B.

| | A₁ | τ₁ | A₂ | τ₂ |
|---|---|---|---|---|
| 0 bp | 0.64 | 3.40 ns | 0.36 | 0.29 ns |
| +1 bp | 0.64 | 3.40 ns | 0.36 | 0.29 ns |
| +2 bp | 0.55 | 2.71 ns | 0.45 | 0.28 ns |
| +3 bp | 0.46 | 1.98 ns | 0.54 | 0.20 ns |
| +4 bp | 0.39 | 1.66 ns | 0.61 | 0.17 ns |
| +5 bp | 0.36 | 1.89 ns | 0.64 | 0.22 ns |
| −2 bp | 0.48 | 2.36 ns | 0.52 | 0.20 ns |
| −3 bp | 0.46 | 2.16 ns | 0.54 | 0.19 ns |
| −4 bp | 0.52 | 2.21 ns | 0.48 | 0.20 ns |
| −5 bp | 0.55 | 2.32 ns | 0.45 | 0.21 ns |
| Mon. | 0.60 | 2.10 ns | 0.40 | 0.64 ns |

To gain further insight into the nature of the excited states, ultrafast transient absorption spectra were measured for the 0 bp and +4 bp dimers, which show the longest and shortest lifetimes, respectively (FIGS. 5A-5B). Within the spectral region probed, both dimers showed negative peaks from ground state bleach/stimulated emission signal at the 0-0 and 0-1 transitions. The decay of the negative peaks was well fit with biexponential kinetics (FIG. 5C) where short components of 145 ps and 330 ps and long components of 4 ns and 1.7 ns were extracted for 0 bp and +4 bp, respectively. The short components may reflect a subpopulation of J-type dimers or aggregation induced nonradiative decay, which was observed previously and assigned to a torsional motion. The long components likely arise from H-type dimers, where the slower timescale for the 0 bp dimer indicates stronger electronic coupling consistent with the conclusions from the steady-state spectra and fluorescence lifetimes.

Global analysis using a two-component parallel decay model was used to extract the decay associated spectra (DAS) for both kinetic components, as shown in FIG. 5D. While both the 0-0 and 0-1 peaks are present in all the DAS, the 0-0/0-1 band ratio was larger for the short component DAS than for the long component DAS. The larger ratio is consistent with the assignment of the short component to sub-populations with weaker H-type electronic coupling and/or even J-type coupling. The smaller 0-0/0-1 band ratio of the long component DAS is indicative of the strong H-type electronic coupling in this sub-population. The 0-0/0-1 band ratio for both DAS of the 0 bp dimer is smaller than for the +4 bp dimer, as expected for the stronger H-type electronic coupling in the 0 bp dimer. Collectively, these variations in the excited-state dynamics establish the ability of the complementary strand to induce changes in geometry and provide flexible building blocks for light harvesting, computing, and imaging applications.

Environmental Sensitivity of Dimer Constructs

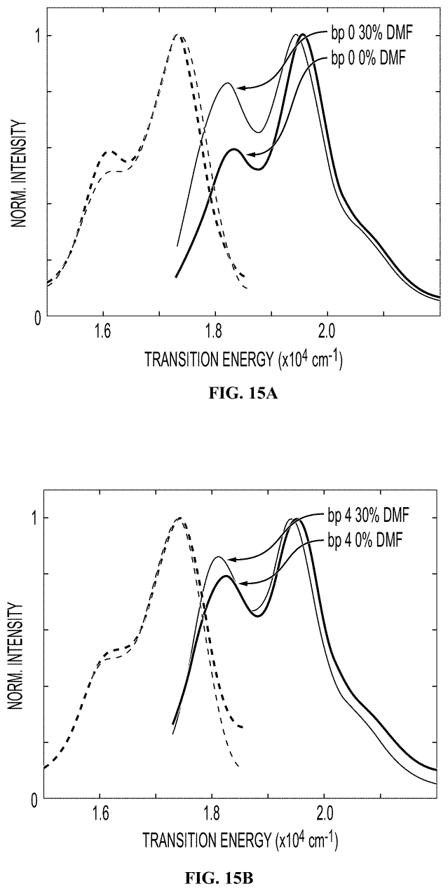

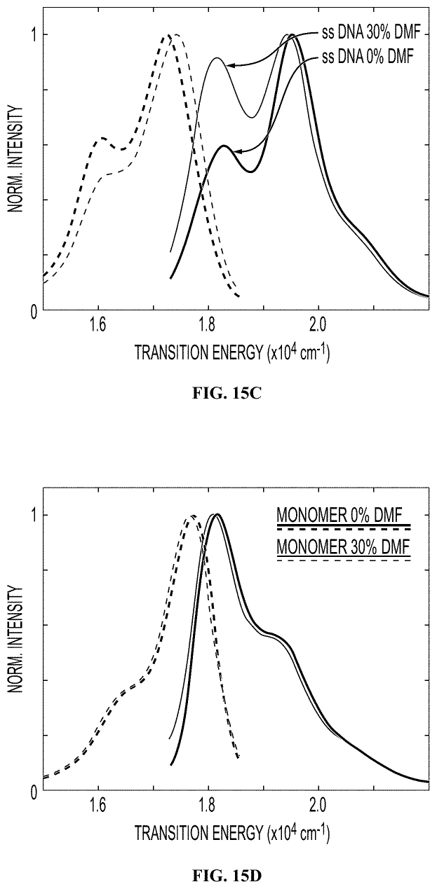

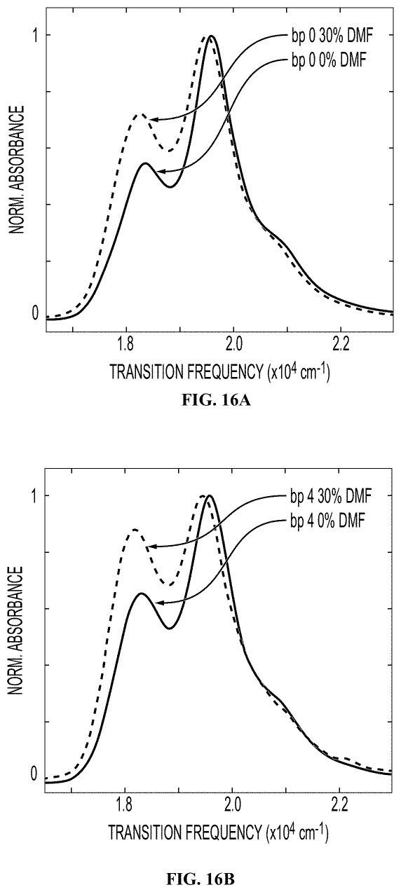

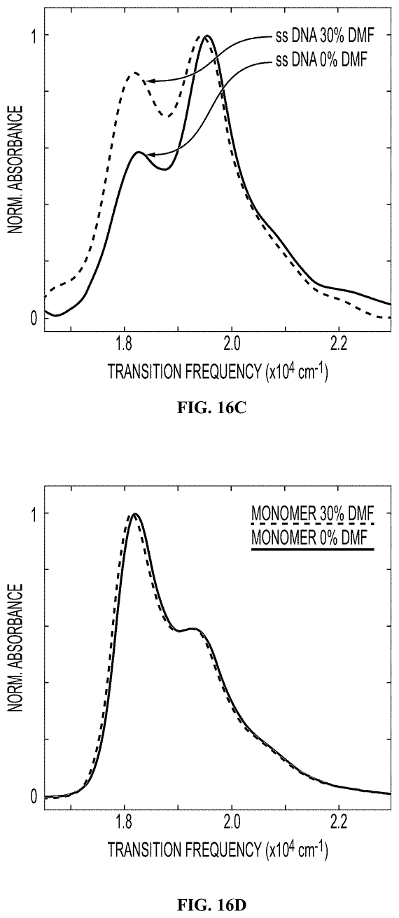

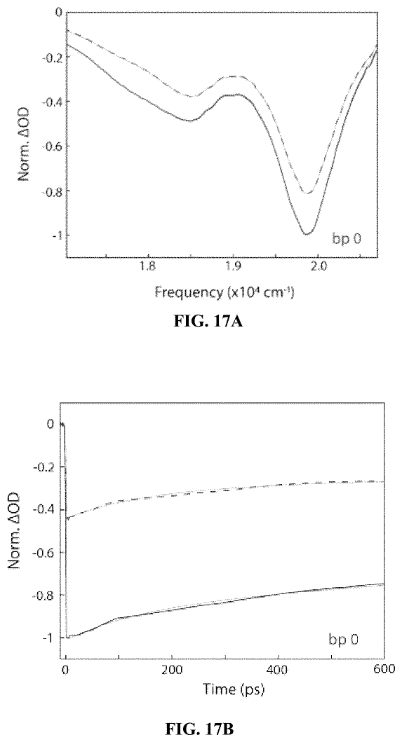

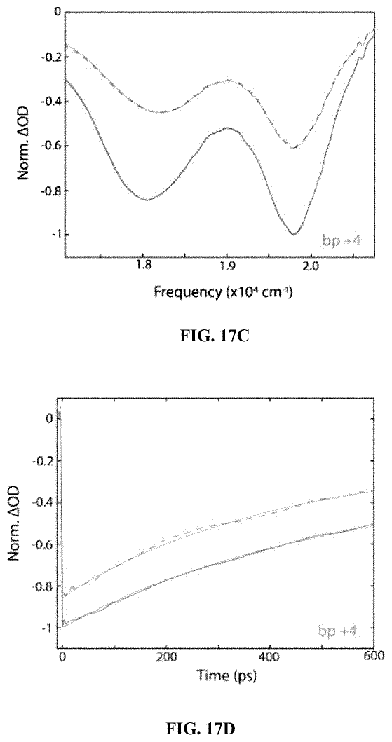

One method for chemical sensing exploits the environmental sensitivity of fluorescence as a reporter of the local composition. Cy3 is likely to exhibit such sensitivity as it is highly susceptible to environmental changes due to its hydrophobic regions and propensity for photoisomerization. The environmental sensitivity of the Cy3 dimers was investigated by measuring the impact of solvent polarity on their emission. The single-stranded, 0 bp and +4 bp dimers were characterized by comparing a buffered solution of 70% water and 30% dimethyl formaldehyde (DMF) and in buffered aqueous solution. The fluorescence excitation spectra (FIGS. 15A-15D) report on the relative contributions of the optical transitions to emission. The difference excitation spectra between 0% DMF and 30% DMF are shown for all three samples in FIG. 7A. The oscillator strength was redistributed from the 0-1 to the 0-0 band and approaches the monomer spectrum, indicating a larger population of weakly coupled dimers in the less polar environment. Similar changes were also observed in the absorption spectra (FIGS. 16A-16D). The transition dipole moment is localized on a primarily hydrophobic region, which may lead to weaker inter-dye interaction in the less polar environment.

The average lifetimes for the three samples are shown in FIG. 7B. The average lifetime of the single-stranded DNA was the longest at 2.2 ns, and the lifetimes of 0 bp and +4 bp DX tiles shortened to 2.0 ns and 1.2 ns, respectively. In the 30% DMF solution, all the lifetimes shortened with the largest change for the single-stranded DNA and the smallest for the +4 bp dimer (FIGS. 7B-7C). In contrast, the lifetime of the monomer increased slightly, similar to previous work on the polarity dependent behavior of cyanine dyes.

The large change for single-stranded DNA likely arises from the lack of structural constraints, which allows for the closest co-facial stacking, and thus the longest lifetime in aqueous solution, as well as the easiest formation of monomer-like population, which shortens the lifetime in 30% DMF. The small change for the +4 bp dimer is likely due to the longer staple strand pulling the dimers apart, resulting in an extended configuration with less co-facial stacking and less freedom to adopt a monomer-like configuration. Consistent with this picture, the +4 bp dimer has a shorter lifetime in 100% aqueous solution, i.e., smaller H-type coupling, and a minimal decrease in lifetime upon addition of 30% DMF, i.e., limited structural rearrangement. Overall, these observations of a polarity-based solvent dependence establish a mechanism to readout the properties of the local environment.

Probes for Fluorescence Lifetime Imaging

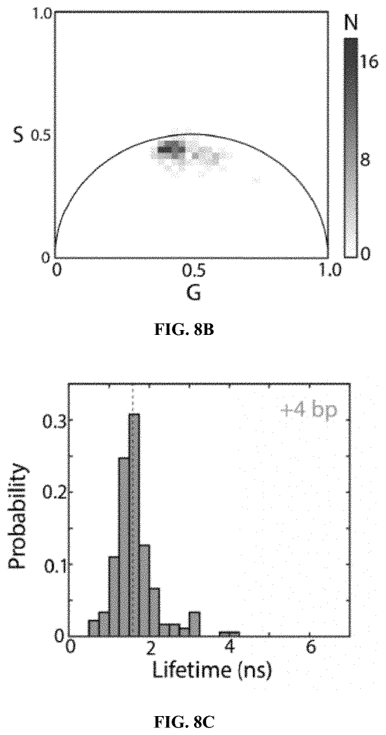

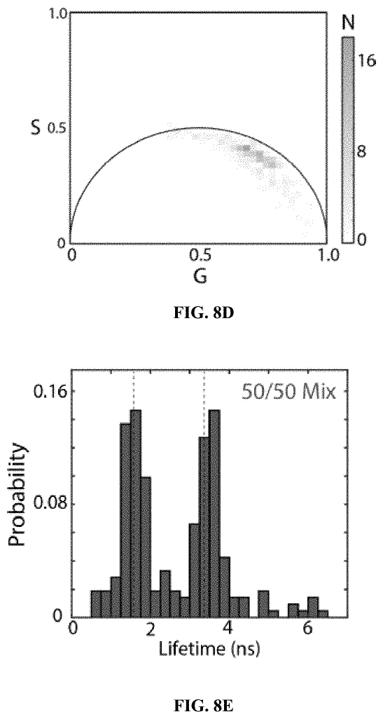

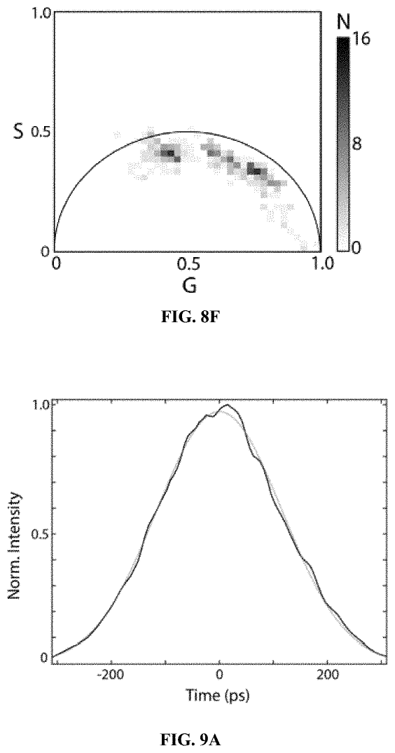

Fluorescence lifetime imaging (FLIM) is widely used to map out biological and materials systems based on differences in the lifetime of fluorescent probes or the material itself. This imaging modality requires distinguishability between lifetimes with high sensitivity and fidelity. While this has typically been achieved via nuclear modification of chromophore monomers, the large (˜65%) variation in lifetime across the series of Cy3 dimers shows that tuning intermolecular interactions, as done here, provides an alternate set of FLIM probes for multiplexed imaging. To test the distinguishability of these constructs in FLIM, confocal single-molecule spectroscopy was performed on the 0 bp Cy3 dimer, +4 bp Cy3 dimer, and a 50%/50% mixture of the two dimer constructs. For each sample, the emission of individual Cy3 dimers was recorded, and the lifetime decay curves were fit to a monoexponential function. The extracted timescales were used to construct lifetime histograms as shown in FIGS. 8A, 8C, and 8E. The mean lifetimes were 3.35 and 1.65 ns for the 0 bp and +4 bp dimers, respectively, consistent with the long-lifetime component from the ensemble measurements. The short-lifetime component observed in the ensemble is absent from the single-molecule data due to the longer instrument response function of the high-sensitivity detector.

The standard deviations of the distributions for both the 0 bp and +4 bp dimers were ˜0.5 ns, which is much less than the separation between the mean lifetime of the two samples (3.35 ns and 1.65 ns, respectively). These results demonstrate that the 0 bp and +4 bp dimers can be easily distinguished in FLIM imaging. As further confirmation of their distinguishability, the data from the 50%/50% mixture was also analyzed. The histogram of the mixture shows a clear bimodal structure (FIG. 8E), confirming the two probes can be resolved in FLIM even with the low signal levels intrinsic to single-molecule imaging.

FLIM experiments are typically performed in the frequency domain for high throughput data collection. To verify distinguishability of the dimers in the frequency domain, a phasor analysis was performed on the time domain data, which is a Fourier transform of the decay curves. The Fourier transformed data is plotted as a function of the real component, G, and the imaginary component, S, of the frequency domain response as shown in FIGS. 8B, 8D, and 8F. The lifetime is proportional to the ratio of S to G in the frequency domain signal, and thus each distribution of single molecules corresponds to distinct populations in the phase space maps (FIGS. 8B and 8D). The center of the 0 bp distribution appears at (G=0.45, S=0.43), while the center of the +4 bp distribution occurs at (G=0.70, S=0.37). Although the +4 bp distribution is broader, the 0 bp and +4 bp dimers are confined above and below G=0.5, respectively. Similar to the time domain results, the distribution of the 50%/50% mixture in phase space has two clear populations separated by a region with a near zero population (FIG. 8E), establishing that these two constructs are also distinguishable in the frequency domain.

The distinguishability of the Cy3 dimers establishes their utility as FLIM probes. Furthermore, the changes in emission spectra and lifetime with solvent polarity allows the dimers to report on their local environment within a FLIM/multiplexed measurement. Indeed, FLIM probes are often used to detect changes in chemical environment, including polarity as well as pH, analyte concentration, and viscosity, which were previously established to also change the Cy3 emission. The staple-strand dependence of the lifetime also al-lows for facile tracking of strand displacement reactions. Precise knowledge of the kinetics of these reactions is often exploited to deploy DNA in analyte sensing. Beyond strand displacement reactions, the dependence of the photo-physics on the complementary staple strand has the potential to enable studies of DNA hybridization and other properties of nucleic acids. Finally, the DNA scaffold means that the construct is biocompatible and easily functionalized for specific conjugation to desired targets.

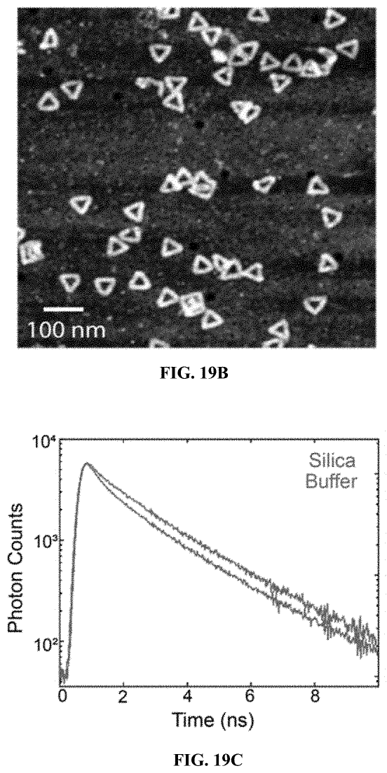

The DNA-scaffolded Cy3 dimers can be impacted by silica coating, as shown in FIGS. 19A-19C. This serves as an orthogonal method of controlling the excited state lifetime based on changes to the DNA environment and can be used to stabilize structures for imaging purposes.

Here, DNA-scaffolded Cy3 dimers and a method to easily tune the photo-physics of the DNA-scaffolded Cy3 dimers via the complementary staple strand. Based on the length of the strand, the DNA scaffold pushed or pulled the dimers, changing the geometry and as a result changing the transition energies, fluorescence lifetime, and solvent dependence of the excited states. In particular, the solvent dependent lifetimes and distinguishability between dimers of different geometries at the single-molecule level demonstrate these constructs can serve as FLIM probes. Overall, the photophysical variations generate a rich collection for designer excited state dynamics, which can be used in fluorescence assays, light harvesting devices, and molecular electronics.

### REFERENCES

1. Lichtman and Conchello, Nature Methods 2005, 2, 910-919.
2. Wang, et al., Analytical Chemistry 2013, 85, 12182-12188.
3. Yuan, et al., Chemical Society Reviews 2013, 42, 622-661.
4. Banal, et al., Nature Materials 2021, 1-9.
5. Mottaghi and Dwyer, Advanced Materials 2013, 25, 3593-3598.
6. Blankenship, Molecular Mechanisms of Photosynthesis 2014 John Wiley&Sons.
7. Cheng and Fleming, Annual Reviews of Physical Chemistry 2009, 60, 241-262.
8. Romero, et al., Nature 2017, 543, 355-365.
9. Bredas, et al., Nature Materials 2017, 16, 35-44.
10. Schroter, et al., Physics Reports 2015, 567, 1-78.
11. Fleming, et al., Faraday Discussions 2012, 155, 27-41.
12. Delor, et al., Journal of the American Chemical Society 2018, 140, 6278-6287.
13. Yang, et al., ACS Sensors 2018, 3, 903-919.
14. Teles and Fonseca, Talanta 2008, 77, 606-623.
15. Scholes and Rumbles, Materials For Sustainable Energy: A Collection of Peer-Reviewed Research and Review Articles from Nature Publishing Group 2011, 12-25.
16. Fassioli, et al., Journal of The Royal Society Interface 2014, 11, 20130901.
17. Scholes, et al., Nature 2017, 543, 647-656.
18. Castellanos, et al., Physical Chemistry Chemical Physics 2020, 22, 3048-3057.
19. Garo and Haner, Angewandte Chemie 2012, 51, 916-919.
20. Dutta, et al., Journal of the American Chemical Society 2011, 133, 11985-11993.
21. Ensslen and Wagenknecht, Accounts of Chemical Research 2015, 48, 2724-2733.
22. Melinger, et al., The Journal of Physical Chemistry B 2016, 120, 12287-12292.
23. Boulais, et al., Nature Materials 2018, 17, 159-166.
24. Wamhoff, et al., Annual Review of Biophysics 2019, 48, 395-419.
25. Huff, et al., The Journal of Physical Chemistry Letters 2019, 10, 2386-2392.
26. Cunningham, et al., The Journal of Physical Chemistry B 2018, 122, 5020-5029.
27. Cunningham, et al., The Journal of Chemical Physics 2017, 147, 055101.
28. Cannon, et al., The Journal of Physical Chemistry A 2017, 121, 6905-6916.
29. Markova, et al., Chemical Communications 2013, 49, 5298-5300.
30. Mathur, et al., The Journal of Physical Chemistry C 2020,
31. Sohail, et al., Chemical Science 2020, 11, 8546-8557.
32. Kringle, et al., Journal of Physical Chemistry 2018, 148, 085101.
33. Yang, et al., Analytical Methods 2017, 9, 1976-1990.
34. Tan, et al., Journal of the American Chemical Society 2011, 133, 2664-2671.
35. Walter, et al., Nano Letters 2017, 17, 2467-2472.
36. Dai, et al., Chemical Communications 2010, 46, 1221-1223.
37. Chen, et al., Sensors and Actuators B: Chemical 2015, 221, 328-333.
38. Cannon, et al., ACS Photonics 2015, 2, 398-404.
39. Kellis, et al., New Journal of Physics 2015, 17, 115007.
40. Uno, et al., Angewandte Chemie 2009, 121, 7498-7501.
41. Abdollahi, et al., International Journal of Molecular Sciences 2018, 19, 2399.
42. Tseng, et al., Journal of Biomedical Optics 2013, 18, 101309.
43. Qiu, et al., The Journal of Physical Chemistry Letters 2018, 9, 4379-4384.
44. Qiu, et al., Small 2017, 13, 1700332.
45. Guo, et al., Nature Communications 2019, 10, 1-14.
46. Banal, et al., Journal of Physical Chemistry Letters
47. Hart, et al., Chem 2021, 7, 752-773.
48. Zhou, et al., Journal of the American Chemical Society 2019, 141, 8473-8481.
49. Hwang, et al., Tetrahedron Letters 2005, 46, 1475-1477.
50. Seo, et al., Tetrahedron Letters 2006, 47, 4037-4039.
51. Vybornyi, et al., Bioconjugate Chemistry 2014, 25, 1785-1793.
52. Gunther, et al., Nucleic Acids Research 2010, 38, 6526-6532.
53. Ruedas-Rama, et al., The Journal of Physical Chemistry B 2010, 114, 6713-6721.
54. Mao, et al., Nature 1999, 397, 144-146.
55. Spano, Accounts of Chemical Research 2010, 43, 429-439.
56. Hestand and Spano, Chemical Reviews 2018, 118, 7069-7163.
57. Kistler, et al., The Journal of Physical Chemistry B 2012, 116, 77-86.
58. Berova, et al., Circular dichroism: principles and applications John Wiley&Sons, 2000.
59. Huff, et al., The Journal of Physical Chemistry B 2021, ASAP.
60. Cunningham, et al., The Journal of Physical Chemistry B 2020, 124, 8042-8049.
61. Wagner, Molecules 2009, 14, 210-237.
62. Er, et al., Chemical Science 2013, 4, 2168-2176.
63. Shvadchak, et al., Biochimica et Biophysica Acta (BBA)-Biomembranes 2017, 1859, 852-859.
64. Zanetti-Domingues, et al., PloS one 2013, 8, e74200.
65. Sanborn, et al., The Journal of Physical Chemistry B 2007, 111, 11064-11074.
66. Pace, et al., The Journal of Physical Chemistry Letters 2021, 12, 8963-8971.
67. Lee, et al., Journal of Photochemistry and Photobiology A: Chemistry 2008, 200, 438-444.
68. Lakner, et al., Scientific Reports 2017, 7, 1-11.
69. Gatzogiannis, et al., Chemical Communications 2012, 48, 8694-8696.
70. tefl, et al., Analytical Biochemistry 2011, 410, 62-69.
71. Martelo, et al., The Journal of Physical Chemistry B 2015, 119, 10267-10274.
72. Szmacinski, et al., Journal of Biomedical Optics 2014, 19, 046017.
73. Qian and Winfree, Science 2011, 332, 1196-1201.
74. Zhu, et al., Nano letters 2021, 21, 1368-1374.
75. Israels, et al., The Journal of Physical Chemistry B 2021, 125, 9426-9440.
76. Magde, et al., Photochemistry and Photobiology 2002, 75, 327-334.
77. Son, et al., Optics Express 2017, 25, 18950-18962.
78. Hua, et al., Nature Methods 2014, 11, 1233-1236.
79. Kondo, et al., Nature Chemistry 2017, 9, 772.
80. Maus, et al., Analytical Chemistry 2001, 73, 2078-2086.
81. Santra, et al., The Journal of Physical Chemistry B 2016, 120, 2484-2490.
82 Szmacinski, et al., Journal of Biomedical Optics 2014, 19, 046017.
83 Lakner, et al., Scientific Reports 2017, 7, 1-11.
84 Martelo, et al., The Journal of Physical Chemistry B 2015, 119, 10267-10274.
85 tefl, et al., Analytical Biochemistry 2011, 410, 62-69.

We claim:

1. A nucleic acid-chromophore comprising a nucleic acid strand comprising two or more adjacent chromophores, wherein, when the nucleic acid-chromophore is in a nucleic acid assembly, one or more photophysical properties of the adjacent chromophores is altered by a change in the nucleic acid assembly,
optionally wherein the nucleic acid assembly comprises a nucleic acid scaffold, and
wherein the change in the nucleic acid assembly is a change in the length of a nucleic acid hybrid in the nucleic acid scaffold that is opposite the adjacent chromophores.

2. The nucleic acid-chromophore of claim 1, wherein the nucleic acid assembly comprises a DX tile,
wherein the change in the nucleic acid assembly is a change in the length of a nucleic acid hybrid in the DX tile opposite the adjacent chromophores.

3. The nucleic acid-chromophore of claim 1, wherein one or more of the chromophores is selected from the group consisting of a cyanine, a squaraine, a pentacene, and a perylene diimide.

4. The nucleic acid-chromophore of claim 1, wherein the adjacent chromophores are cyanines,
optionally wherein the cyanines are selected from the group consisting of indocarbocyanines, indodicarbocyanines and indotricarbocyanines.

5. The nucleic acid-chromophore of claim 1, wherein the nucleic acid strand is composed of one or more selected from the group consisting of deoxyribonucleotides (DNA), ribonucleotides (RNA), locked nucleic acids (LNA), peptide nucleic acids (PNA) and analogs or modified nucleotides thereof.

6. The nucleic acid-chromophore of claim 1, wherein the nucleic acid-chromophore is in a nucleic acid assembly, and
wherein the nucleic acid assembly is coupled to a biological or nonbiological material.

7. The nucleic acid-chromophore of claim 6, wherein the nucleic acid assembly is operably coupled to the biological or nonbiological material, and
wherein interaction of a molecule of interest with the biological or nonbiological material produces the change in the nucleic acid assembly.

8. The nucleic acid-chromophore of claim 1, wherein the one or more photophysical properties of the adjacent chromophores is selected from the group consisting of the quantum yield of the adjacent chromophores, the energy dependent optical density of the adjacent chromophores, the emission energetic profile of the adjacent chromophores, and the excited state lifetime of the adjacent chromophores.

9. The nucleic acid-chromophore of claim 1, wherein the one or more photophysical properties of the adjacent chromophores comprises the excited state lifetime of the adjacent chromophores, and
wherein the excited state lifetime of the adjacent chromophores is altered by a change in solvent polarity.

10. The nucleic acid-chromophore of claim 1, wherein the alteration in the one or more photophysical properties of the adjacent chromophores is sufficient to distinguish a single altered nucleic acid assembly from a single unaltered nucleic acid assembly.

11. The nucleic acid-chromophore of claim 1, wherein the nucleic acid assembly is encapsulated.

12. The nucleic acid-chromophore of claim 1, wherein the nucleic acid assembly is encapsulated in an organic, or in an inorganic material, or in a combination of organic and inorganic material.

13. The nucleic acid-chromophore of claim 12, wherein the inorganic material is silica.

14. A method of detecting a change in a nucleic acid assembly, wherein the nucleic acid assembly comprises the nucleic acid-chromophore of claim 1,
wherein the method comprises measuring one or more of the photophysical properties of the adjacent chromophores, and
wherein the change is detected if one or more of the measured photophysical properties of the adjacent chromophores is altered compared to a reference photophysical property.

15. The method of claim 14, wherein the reference photophysical property is the photophysical property of the nucleic acid assembly in the absence of a change in the nucleic acid assembly.

16. The method of claim 14, wherein the change in the nucleic acid assembly is produced by interaction of a molecule of interest with a biological or nonbiological material to which the nucleic acid assembly is operably coupled, or by correct assembly of a scaffold origami of which the nucleic acid assembly becomes a part, or by a change in the milieu of the nucleic acid assembly.

17. The method of claim 16, wherein the change in milieu of the nucleic acid assembly is a change in the polarity of solvent in which the nucleic acid assembly is dissolved or suspended.

18. A method of multiplex detection, the method comprising:
(a) labeling each of a first plurality of different targets of interest with the same first nucleic acid-chromophore of claim 1,
wherein each of the first nucleic acid-chromophores labeling each of the plurality of different targets of interest is in a different nucleic acid assembly,
wherein each of the different nucleic acid assemblies has a change relative to the other nucleic acid assemblies such that one or more of the photophysical properties of the adjacent chromophores is altered relative to those photophysical properties of the adjacent chromophores in the other nucleic acid assemblies, and
(b) detecting the photophysical properties of the adjacent chromophores in the different nucleic acid assemblies, thereby detecting the different targets of interest of the first plurality of different targets of interest.

19. The method of claim 18 further comprising:
(c) labeling each of a second plurality of different targets of interest with the same second nucleic acid-chromophore of claim 1,
wherein each of the second nucleic acid-chromophores labeling each of the second plurality of different targets of interest is in a different nucleic acid assembly,
wherein each of the different nucleic acid assemblies has a change relative to the other nucleic acid assemblies such that one or more of the photophysical properties of the adjacent chromophores is altered relative to those photophysical properties of the adjacent chromophores in the other nucleic acid assemblies, and
(d) detecting the photophysical properties of the adjacent chromophores in the different nucleic acid assemblies, thereby detecting the different targets of interest of the second plurality of different targets of interest.

20. A method of altering one or more photophysical properties of a nucleic acid-chromophore, the method comprising making a change in a nucleic acid assembly, wherein the nucleic acid assembly comprises the nucleic acid chromophore,
wherein the nucleic acid chromophore comprises a nucleic acid strand comprising two or more adjacent chromophores, and
wherein the one or more photophysical properties of the nucleic acid-chromophore are altered by the change in the nucleic acid assembly,
optionally wherein the nucleic acid assembly comprises a nucleic acid scaffold, and
wherein the change in the nucleic acid assembly is a change in the length of a nucleic acid hybrid in the nucleic acid scaffold that is opposite the adjacent chromophores.

21. The method of claim 20, wherein the nucleic acid assembly comprises a DX tile, and
wherein the change in the nucleic acid assembly is a change in the length of a nucleic acid hybrid in the DX tile opposite the adjacent chromophores.

22. The method of claim 20, wherein one or more of the chromophores is a cyanine, a squaraine, a pentacene, or a perylene diimide.

23. The method of claim 20, wherein the adjacent chromophores are cyanines,
optionally wherein the cyanines are selected from the group consisting of indocarbocyanines, indodicarbocyanines and indotricarbocyanines.

24. The method of claim 20, wherein the nucleic acid strand comprises one or more selected from the group consisting of deoxyribonucleotides (DNA), ribonucleotides (RNA), locked nucleic acids (LNA), peptide nucleic acids (PNA) and analogs or modified nucleotides thereof.

25. The method of claim 20, wherein the nucleic acid-chromophore is in a nucleic acid assembly, and
wherein the nucleic acid assembly is coupled to a biological or nonbiological material.

26. The method of claim 25, wherein the nucleic acid assembly is operably coupled to the biological or nonbiological material, and
wherein interaction of a molecule of interest with the biological or nonbiological material produces the change in the nucleic acid assembly.

27. The method of claim 26, wherein the one or more photophysical properties of the adjacent chromophores comprises the quantum yield of the adjacent chromophores.

28. The method of claim 20, wherein the one or more photophysical properties of the adjacent chromophores comprises the energy dependent optical density of the adjacent chromophores, or the emission energetic profile of the adjacent chromophores, or the excited state lifetime of the adjacent chromophores.

29. The method of claim 20, wherein the one or more photophysical properties of the adjacent chromophores comprises the excited state lifetime of the adjacent chromophores,
wherein the excited state lifetime of the adjacent chromophores is altered by a change in solvent polarity.

30. The method of claim 20, wherein the alteration in the one or more photophysical properties of the adjacent chromophores is sufficient to distinguish a single altered nucleic acid assembly from a single unaltered nucleic acid assembly.

31. The method of claim 20, wherein the nucleic acid assembly is encapsulated.

32. The method of claim 20, wherein the nucleic acid assembly is encapsulated in an organic material, or an inorganic material, or both an organic material and an inorganic material.

33. The method of claim 32, wherein the inorganic material is silica.
